(19) United States
(12) Patent Application Publication
(10) Pub. No.: US 2025/0289847 A1
(43) Pub. Date: Sep. 18, 2025

(54) SYSTEMS AND METHODS FOR BIOMOLECULE COLLECTION AND ISOLATION

(71) Applicant: LIFE TECHNOLOGIES CORPORATION, Carlsbad, CA (US)

(72) Inventors: Mark Thomas Smith, Nibley, UT (US); Portia Christina Densley, Logan, UT (US); Natraj Ramasubramanyan, Westborough, MA (US)

(21) Appl. No.: 19/077,328

(22) Filed: Mar. 12, 2025

Related U.S. Application Data
(60) Provisional application No. 63/565,369, filed on Mar. 14, 2024.

(51) Int. Cl. C07K 1/22; C07H 1/06; C07K 1/36; C08B 37/00

(52) U.S. Cl. CPC C07K 1/22; C07H 1/06; C07K 1/36; C08B 37/00

(57) ABSTRACT

Systems and methods for enriching or isolating one or more biomolecules are disclosed herein. Existing biomolecule collection and isolation systems and methods require multiple, sequential processes for pre-processing biomolecules after in vitro production and before final isolation (e.g., using affinity chromatography). Failure to sufficiently pre-process (e.g., purify) biomolecules prior to isolation using affinity chromatography frequently results in failure of chromatography equipment, unacceptable decreases in contaminant breakthrough, process throughput, and reagent usage, and increased likelihood of sample contamination or degradation. Systems and methods are disclosed herein that eliminate one or more steps of existing biomolecule isolation systems and protocols, which can decrease time and cost of purification or isolation of biomolecules of interest while maintaining acceptable yield and purity parameters.

### CROSS-REFERENCE TO RELATED APPLICATIONS

This application claims the benefit of U.S. Provisional Application No. 63/565,369, filed on Mar. 14, 2024, which is incorporated herein by specific reference.

### BACKGROUND

Large-scale biomolecule production frequently involves multiple stages of purification, enrichment, and/or isolation of the biomolecules from the carrier in which the biomolecules of interest are produced. For example, existing systems and methods for purifying biomolecules following in vitro production in bioreactors relies upon separate depth filtration, sterile sample holding, and affinity chromatography operational steps to achieve sufficient biomolecule purity and yield for laboratory or clinical use. However, the multiplicity and limited throughput of these extra processing steps can have severe adverse effects on cost efficiency of the biomolecule production process.

Depth filtration, in particular, is a major bottleneck in the process of biomolecule production. In a depth filtration process step, an unclarified sample containing a biomolecule of interest is passed through a depth filter, which comprises a series of filter meshes having progressively smaller pore sizes. In many cases, the clarification process of existing systems and methods involves several different sequential unit operation steps necessary to complete the clarification and filtration of the sample before it can be applied to the chromatography unit (e.g., chromatography column). For example, existing technologies commonly require a primary clarification involving depth filtration of the sample after the sample is received from a bioreactor, followed by a secondary clarification involving a second round of depth filtration, then a sterile filtration unit operation, which is often followed by a product hold step, which may be followed by a second sterile filtration step before the sample is applied to the chromatography unit. As particulates and other contaminants are captured in the depth filter's meshes, resistance to flow through the depth filter increases. The resulting progressive slowing of filtration rate during biomolecule purification in a depth filter makes continuous flow through existing biomolecule purification systems problematic at large volumes. If pressure is increased, breakthrough of contaminants into the post-filtration sample increases rapidly. Batch processing configurations are often used instead of continuous depth filtration for existing biomolecule purification technologies, as the discretization of throughput volumes in batch processing can allow opportunities to change filter units between batches and as the extended sequence of unit operations is often difficult to perform without a product hold step, without 24-hour technician support of the process. Regardless of whether batch processing is used, however, the need to frequently replace the costly depth filters greatly increases the time and cost required for performing biomolecule purification using existing technologies.

Attempting to remove the depth filtration step from existing biomolecule purification systems and methods is problematic as well. In such cases, unclarified carrier medium comprising the biomolecule of interest quickly clogs the affinity chromatography unit, which stops flow through the column and causes an increase in pressure in the column. If additional pressure is applied, the column will fail, resulting in loss of biomolecules into the waste stream and an unacceptable increase in concentration of contaminants in the collected eluent.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIGS. 1A-1F show schematics of bioprocessing systems (e.g., biomolecule collection systems), in accordance with embodiments;

FIG. 2A shows a schematic of a chromatography unit arranged in an axial flow configuration (AFC);

FIG. 2B shows a schematic of a chromatography unit arranged in a radial flow configuration (RFC), in accordance with embodiments;

FIGS. 3 and 4 show steps of methods for enriching a biomolecule of interest, in accordance with embodiments;

FIG. 5A shows measured sample pressure in chromatography columns plotted against processed volume of unclarified samples, in accordance with embodiments;

FIG. 5B shows measured sample pressure in the chromatography column of biomolecule collection systems comprising pre-column centrifugation disclosed herein plotted against processed volume of unclarified samples, in accordance with embodiments;

FIG. 6A shows pressure data in chromatography columns of biomolecule collection systems using affinity beads having diameters between 50 and 100 microns, in accordance with embodiments;

FIG. 6B shows pressure data in chromatography columns of biomolecule collection systems using affinity beads having diameters between 100 and 200 microns, in accordance with embodiments;

FIG. 7 shows percent recovery data for repeated use of chromatography columns, in accordance with embodiments.

FIG. 8 shows breakthrough curves for biomolecule collection systems of various configurations with or without a wash cycle, in accordance with embodiments;

FIG. 9 shows breakthrough curves for biomolecule collection systems of various configurations, in accordance with embodiments;

FIG. 10 shows diagrams representing processes associated with various configurations of biomolecule collection systems, in accordance with embodiments;

The figures may not be to scale in absolute or comparative terms and are intended to be exemplary. The relative placement of features and elements may have been modified for the purpose of illustrative clarity. Where practical, the same or similar reference numbers denote the same or similar or equivalent structures, features, aspects, or elements, in accordance with one or more embodiments.

### SUMMARY

In some embodiments, a system for separating a substance of interest of an unfiltered fluid sample comprises: a centrifuge unit; and a chromatography unit comprising a plurality of beads, each of the plurality of beads comprising a capture ligand; and a fluidic apparatus configured to transfer the fluid sample after centrifugation from the centrifuge unit to the chromatography unit. In some embodiments, each of the plurality of beads has a diameter of less than 200 micrometers. In some embodiments, a system for separating a substance of interest of an unfiltered fluid sample comprises: a centrifuge; and a chromatography unit in fluid communication with the centrifuge, wherein the chromatography unit is configured to receive an unfiltered supernatant comprising the substance of interest from the centrifuge unit without the supernatant passing through a filter. In some embodiments, the chromatography unit is an affinity chromatography column. In some embodiments, the affinity chromatography column comprises a plurality of beads, each of the plurality of beads comprising a capture ligand. In some embodiments, the capture ligand is protein A. In some embodiments, the capture ligand is an adeno-associated virus ligand. In some embodiments, the affinity chromatography column is configured to capture a molecule of interest selected from a protein, a carbohydrate, a lipid, or a nucleic acid. In some embodiments, the system does not comprise a filter between the centrifuge unit and the chromatography unit. In some embodiments, each of the plurality of beads has a diameter of from 50 micrometers to 100 micrometers or from 100 micrometers to 190 micrometers. In some embodiments, the chromatography unit is configured as an axial flow column (AFC) or a radial flow column (RFC). In some embodiments, the chromatography unit has a void volume fraction of 10% to 20%, 20% to 30%, 30% to 40%, or up to 50%. In some embodiments, the system is a closed system. In some embodiments, the system is configured to maintain sterility of the substance of interest by avoiding external contaminants. In some embodiments, the centrifuge unit comprises an inlet tube configured to receive the fluid sample. In some embodiments, the system further comprises a pump configured to transfer fluid from the centrifuge unit to the chromatography unit. In some embodiments, the system further comprises a hold vessel, wherein the hold vessel comprises a hold vessel inlet and a hold vessel outlet, the hold vessel inlet in fluid communication with the centrifuge outlet, and the hold vessel outlet in fluid communication with an inlet of the chromatography unit. In some embodiments, the chromatography unit comprises an outlet. In some embodiments, the system further comprises a collection container coupled to the outlet of the chromatography unit. In some embodiments, the outlet is in fluid communication with an inlet of a second chromatography unit. In some embodiments, the system is configured for continuous processing of the fluid sample. In some embodiments, the system is configured for semi-continuous processing of the fluid sample. In some embodiments, the system is configured for batch processing of the fluid sample. In some embodiments, the fluid sample comprises unlysed cells. In some embodiments, the fluid sample comprises lysed cells. In some embodiments, the fluid sample has a turbidity of from about 1.0 Nephelometric Turbidity Units (NTU) to about 100 NTU, from about 100 NTU to about 250 NTU, from about 250 NTU to about 500 NTU, from about 500 NTU to about 750 NTU, from about 750 NTU to about 1,000 NTU, from about 1,000 NTU to about 2,500 NTU, from about 2,500 NTU to about 5,000 NTU, from about 5,000 NTU to about 10,000 NTU, or greater than about 10,000 NTU prior to introduction into the system. In some embodiments, the centrifuged sample has a turbidity of from about 0.1 Nephelometric Turbidity Units (NTU) to about 1.0 NTU, from about 1.0 NTU to about 5.0 NTU, from about 5.0 NTU to about 20 NTU, from about 20 NTU to about 50 NTU, from about 50 NTU to about 100 NTU, from about 100 NTU to about 200 NTU, from about 200 NTU to about 300 NTU, from about 300 NTU to about 500 NTU, or greater than about 500 NTU after centrifugation. In some embodiments, an eluent collected from the chromatography unit has a turbidity of from about 0.1 Nephelometric Turbidity Units (NTU) to about 0.01 NTU, from about 0.5 NTU to about 1.0 NTU, from about 1.0 NTU to about 2.5 NTU, from about 2.5 NTU to about 5.0 NTU, from about 0.01 NTU to about 5.0 NTU, from about 0.5 NTU to about 5.0 NTU, from about 1.0 NTU to about 5.0 NTU, from about 2.5 NTU to about 5.0 NTU, or greater than about 5.0 NTU. In some embodiments, the fluid sample has a substance of interest concentration before centrifugation of from about 1 grams/liter (g/L) to about 10 g/L of substance of interest, from about 10 g/L to about 25 g/L, from about 25 g/L to about 30 g/L, from about 30 g/L to about 35 g/L, or greater than 35 g/L. In some embodiments, a loading capacity of the fluid sample is at least 90% of a loading capacity of a depth filtered fluid sample when loaded onto the chromatographic unit. In some embodiments, the yield recovery of the substance of interest in the centrifuged sample after processing with the centrifugation unit, relative to the amount of substance of interest in the fluid sample when the fluid sample is introduced to the centrifugation unit, is at least about 75%, at least about 80%, at least about 85%, at least about 90%, or at least about 95%. In some embodiments, the yield recovery of the substance of interest after processing with the chromatography unit, relative to the amount of substance of interest in the centrifuged sample when the centrifuged sample is introduced to the chromatography unit, is at least about 75%, at least about 80%, at least about 85%, at least about 90%, or at least about 95%. In some embodiments, the yield recovery of the substance of interest after processing with the chromatography unit, relative to the amount of substance of interest in the fluid sample when the fluid sample is introduced to the centrifugation unit, is at least about 60%, at least about 70%, at least about 80%, at least about 90%, or at least about 95%. In some embodiments, the system maintains a pressure within the chromatography unit of 1.0 MPa or less during processing of at least 50 L of centrifuged sample, at least 100 L of centrifuged fluid sample, at least 150 L of centrifuged fluid sample, at least 200 L of centrifuged fluid sample, at least 300 L of centrifuged fluid sample, at least 400 L of centrifuged fluid sample, at least 500 L of centrifuged fluid sample, at least 600 L of centrifuged fluid sample, at least 700 L of centrifuged fluid sample, or at least 800 L of centrifuged fluid sample. In some embodiments, the system is configured to process at least 2,000 L of the substance of interest with the centrifuge unit and the chromatography unit in less than 40 hours, less than 35 hours, less than 30 hours, less than 25 hours, less than 24 hours, less than 20 hours, less than 18 hours, or less than 15 hours. In some embodiments, a concentration of the substance of interest in an eluent at the outlet of the chromatography unit is at least 50% greater, from 50% to 100% greater, from 100% to 150% greater, from 150% to 200% greater, from 200% to 250% greater, or more than 250% greater than a concentration of the substance of interest in the fluid sample at the inlet of the centrifuge.

In some embodiments, a method for collecting a substance of interest comprises: centrifuging a fluid sample comprising the substance and a cellular component, the cellular component; transferring the centrifuged fluid sample to a chromatography unit without filtering the sample; and separating the substance from the fluid sample using the chromatography unit. In some embodiments, the method further comprises washing the fluid sample after transferring the fluid sample to the chromatography unit. In some embodiments, the fluid sample is washed two or more times after transferring the fluid sample to the chromatography unit. In some embodiments, the fluid sample is washed with a solution comprising one or more mixture selected from: a. 5% to 20% isopropyl alcohol and 0.2 molar (M) to 1 M arginine; b. 5% to 20% isopropyl alcohol, 1 M to 3 M urea, and 0.5% to 2% polysorbate 20; c. 50 mM to 500 mM Tris, 0.1 M to 1 M sodium chloride; or d. 50 mM to 500 mM Tris. In some embodiments, the fluid sample is washed with a solution comprising one or more mixture selected from: a. 5% to 20% isopropyl alcohol and 0.2 molar (M) to 1 M arginine; b. 5% to 20% isopropyl alcohol, 1 M to 3 M urea, and 0.5% to 2% polysorbate 20; c. 50 mM to 500 mM Tris, 0.1 M to 1 M sodium chloride at pH 7.5; or d. 50 mM to 500 mM Tris at pH 7.5. In some embodiments, the method further comprises eluting the substance of interest after transferring the fluid sample to the chromatography unit. In some embodiments, the method further comprises transferring an eluent comprising the eluted substance of interest to a second chromatography unit after eluting the fluid sample, wherein the eluent is not filtered prior to being received at the second chromatography unit. In some embodiments, the chromatography unit is an affinity chromatography column. In some embodiments, the affinity chromatography column comprises a plurality of beads, each of the plurality of beads comprising a capture ligand. In some embodiments, the capture ligand is protein A. In some embodiments, the capture ligand is an adeno-associated virus ligand. In some embodiments, the affinity chromatography column is configured to capture a target protein selected from a protein, a carbohydrate, a nucleic acid, or a lipid. In some embodiments, each of the plurality of beads has a diameter of from 50 micrometers to 100 micrometers or from 100 micrometers to 190 micrometers. In some embodiments, each of the plurality of beads has a diameter of from 100 micrometers to 200 micrometers. In some embodiments, the chromatography unit is configured as an axial flow column (AFC) or a radial flow column (RFC). In some embodiments, the chromatography unit has a void volume fraction of from 10% to 20%, 20% to 30%, 30% to 40%, or up to 50%. In some embodiments, the method is performed as a continuous process. In some embodiments, the method is performed as a semi-continuous process. In some embodiments, the method is performed as a batch process. In some embodiments, the fluid sample comprises unlysed cells. In some embodiments, the fluid sample comprises lysed cells. In some embodiments, the fluid sample has a turbidity of from about 1.0 Nephelometric Turbidity Units (NTU) to about 100 NTU, from about 100 NTU to about 250 NTU, from about 250 NTU to about 500 NTU, from about 500 NTU to about 750 NTU, from about 750 NTU to about 1,000 NTU, from about 1,000 NTU to about 2,500 NTU, from about 2,500 NTU to about 5,000 NTU, from about 5,000 NTU to about 10,000 NTU, or greater than about 10,000 NTU prior to centrifuging the fluid sample. In some embodiments, the centrifuged sample has a turbidity of from about 0.1 Nephelometric Turbidity Units (NTU) to about 1.0 NTU, from about 1.0 NTU to about 5.0 NTU, from about 5.0 NTU to about 20 NTU, from about 20 NTU to about 50 NTU, from about 50 NTU to about 100 NTU, from about 100 NTU to about 200 NTU, from about 200 NTU to about 300 NTU, from about 300 NTU to about 500 NTU, or greater than about 500 NTU after centrifuging the fluid sample. In some embodiments, an eluent collected from the chromatography unit has a turbidity of from about 0.1 Nephelometric Turbidity Units (NTU) to about 0.01 NTU, from about 0.5 NTU to about 1.0 NTU, from about 1.0 NTU to about 2.5 NTU, from about 2.5 NTU to about 5.0 NTU, from about 0.01 NTU to about 5.0 NTU, from about 0.5 NTU to about 5.0 NTU, from about 1.0 NTU to about 5.0 NTU, from about 2.5 NTU to about 5.0 NTU, or greater than about 5.0 NTU. In some embodiments, the fluid sample has a substance of interest concentration before centrifugation of from about 1 grams/liter (g/L) to about 10 g/L of substance of interest, from about 10 g/L to about 25 g/L, from about 25 g/L to about 30 g/L, from about 30 g/L to about 35 g/L, or greater than 35 g/L. In some embodiments, a loading capacity of the fluid sample is at least 90% of a loading capacity of a depth filtered fluid sample when loaded onto the chromatographic unit. In some embodiments, the yield recovery of the substance of interest in the centrifuged sample after processing with the centrifugation unit, relative to the amount of substance of interest in the fluid sample when the fluid sample is introduced to the centrifugation unit, is at least about 75%, at least about 80%, at least about 85%, at least about 90%, or at least about 95%. In some embodiments, the yield recovery of the substance of interest after processing with the chromatography unit, relative to the amount of substance of interest in the centrifuged sample when the centrifuged sample is introduced to the chromatography unit, is at least about 75%, at least about 80%, at least about 85%, at least about 90%, or at least about 95%. In some embodiments, the yield recovery of the substance of interest after processing with the chromatography unit, relative to the amount of substance of interest in the fluid sample when the fluid sample is introduced to the centrifugation unit, is at least about 60%, at least about 70%, at least about 80%, at least about 90%, or at least about 95%. In some embodiments, a pressure within the chromatography unit is maintained at 1.0 MPa or less during processing of at least 50 L of centrifuged fluid sample, at least 100 L of centrifuged fluid sample, at least 150 L of centrifuged fluid sample, at least 200 L of centrifuged fluid sample, at least 300 L of centrifuged fluid sample, at least 400 L of centrifuged fluid sample, at least 500 L of centrifuged fluid sample, at least 600 L of centrifuged fluid sample, at least 700 L of centrifuged fluid sample, or at least 800 L of centrifuged fluid sample. In some embodiments, at least 1,000 L of the substance of interest is processed with the centrifuge unit and the chromatography unit in less than 40 hours, less than 35 hours, less than 30 hours, less than 25 hours, less than 24 hours, less than 20 hours, less than 18 hours, less than 15 hours, less than 12 hours, or less than 10 hours. In some embodiments, a concentration of the substance of interest in an eluent at the outlet of the chromatography unit is at least 50% greater, from 50% to 100% greater, from 100% to 150% greater, from 150% to 200% greater, from 200% to 250% greater, or more than 250% greater than a concentration of the substance of interest in the fluid sample at the inlet of the centrifuge.

In some embodiments, a system for filter-free harvest and capture of a substance of interest from a fluid sample comprises: a centrifuge unit; a chromatography unit; and a fluidic apparatus configured to directly transfer the fluid sample after centrifugation from the centrifuge unit to the chromatography unit, wherein the chromatography unit is configured to capture and harvest the substance of interest from the fluid sample. In some embodiments, the system further comprises a housing bounding a chamber and having an inlet port and an outlet port, wherein the centrifuge unit and the chromatography unit are disposed inside the chamber. In some embodiments, the inlet port of the chamber is in fluid communication with an inlet of the centrifuge unit and the outlet port of the chamber is in fluid communication with an outlet of the chromatography unit. In some embodiments, an inlet of the chromatography unit is in fluid communication with a wash reservoir. In some embodiments, the outlet port of the chamber is in fluid communication with a biomolecule collection container. In some embodiments, the outlet port of the chamber is in fluid communication with a waste container. In some embodiments, the chromatography unit is an affinity chromatography column. In some embodiments, the affinity chromatography column comprises a plurality of beads, each of the plurality of beads comprising a capture ligand. In some embodiments, the capture ligand is protein A. In some embodiments, the capture ligand is an adeno-associated virus ligand. In some embodiments, the affinity chromatography column is configured to capture a molecule of interest selected from a protein, a carbohydrate, a lipid, or a nucleic acid. In some embodiments, the system does not comprise a filter between the centrifuge unit and the chromatography unit. In some embodiments, the chromatography unit comprises a plurality of beads, and wherein each of the plurality of beads has a diameter of from 50 micrometers to 100 micrometers or from 100 micrometers to 190 micrometers. In some embodiments, the chromatography unit is configured as an axial flow column (AFC) or a radial flow column (RFC). In some embodiments, the chromatography unit has a void volume fraction of 10% to 20%, 20% to 30%, 30% to 40%, or up to 50%. In some embodiments, the system is a closed system. In some embodiments, the system is configured to maintain sterility of the substance of interest by avoiding external contaminants. In some embodiments, the system further comprising a pump configured to transfer fluid from the centrifuge unit to the chromatography unit. In some embodiments, the system is configured for continuous processing of the fluid sample. In some embodiments, the system is configured for semi-continuous processing of the fluid sample. In some embodiments, the system is configured for batch processing of the fluid sample. In some embodiments, the fluid sample comprises unlysed cells. In some embodiments, the fluid sample comprises lysed cells. In some embodiments, the fluid sample has a turbidity of from about 1.0 Nephelometric Turbidity Units (NTU) to about 100 NTU, from about 100 NTU to about 250 NTU, from about 250 NTU to about 500 NTU, from about 500 NTU to about 750 NTU, from about 750 NTU to about 1,000 NTU, from about 1,000 NTU to about 2,500 NTU, from about 2,500 NTU to about 5,000 NTU, from about 5,000 NTU to about 10,000 NTU, or greater than about 10,000 NTU prior to introduction into the system. In some embodiments, the centrifuged sample has a turbidity of from about 0.1 Nephelometric Turbidity Units (NTU) to about 1.0 NTU, from about 1.0 NTU to about 5.0 NTU, from about 5.0 NTU to about 20 NTU, from about 20 NTU to about 50 NTU, from about 50 NTU to about 100 NTU, from about 100 NTU to about 200 NTU, from about 200 NTU to about 300 NTU, from about 300 NTU to about 500 NTU, or greater than about 500 NTU after centrifugation. In some embodiments, an eluent collected from the chromatography unit has a turbidity of from about 0.1 Nephelometric Turbidity Units (NTU) to about 0.01 NTU, from about 0.5 NTU to about 1.0 NTU, from about 1.0 NTU to about 2.5 NTU, from about 2.5 NTU to about 5.0 NTU, from about 0.01 NTU to about 5.0 NTU, from about 0.5 NTU to about 5.0 NTU, from about 1.0 NTU to about 5.0 NTU, from about 2.5 NTU to about 5.0 NTU, or greater than about 5.0 NTU. In some embodiments, the fluid sample has a substance of interest concentration before centrifugation of from about 1 grams/liter (g/L) to about 10 g/L of substance of interest, from about 10 g/L to about 25 g/L, from about 25 g/L to about 30 g/L, from about 30 g/L to about 35 g/L, or greater than 35 g/L. In some embodiments, a loading capacity of the fluid sample is at least 90% of a loading capacity of a depth filtered fluid sample when loaded onto the chromatographic unit.

In some embodiments, an automated filter-free harvest and capture system comprises: a system controller comprising a processor and memory for storing operational instructions and controlling components of the bioprocessing system; a first valve and a first pump in fluid communication with an outlet of a bioreactor; a first sensor coupled to the outlet of the bioreactor and in electronic communication with the system controller; a housing comprising a chamber with an inlet port, and an outlet port, in fluid communication with the outlet of the bioreactor; the housing comprising: a centrifuge unit; a chromatography unit in fluid communication with the centrifuge unit; a first set of instructions stored in the memory for flowing a fluid sample from the bioreactor through the centrifuge unit and the chromatography unit and to capture and harvest a substance of interest from the fluid sample. In some embodiments, an inlet of the chromatography unit is in fluid communication with a wash reservoir. In some embodiments, the outlet port of the chamber is in fluid communication with a biomolecule collection container. In some embodiments, the outlet port of the chamber is in fluid communication with a waste container. In some embodiments, the chromatography unit is an affinity chromatography column. In some embodiments, the affinity chromatography column comprises a plurality of beads, each of the plurality of beads comprising a capture ligand. In some embodiments, the capture ligand is protein A. In some embodiments, the capture ligand is an adeno-associated virus ligand. In some embodiments, the affinity chromatography column is configured to capture a molecule of interest selected from a protein, a carbohydrate, a lipid, or a nucleic acid. In some embodiments, the system does not comprise a filter between the centrifuge unit and the chromatography unit. In some embodiments, the chromatography unit comprises a plurality of beads, and wherein each of the plurality of beads has a diameter of from 50 micrometers to 100 micrometers or from 100 micrometers to 190 micrometers. In some embodiments, the chromatography unit is configured as an axial flow column (AFC) or a radial flow column (RFC). In some embodiments, the chromatography unit has a void volume fraction of 10% to 20%, 20% to 30%, 30% to 40%, or up to 50%. In some embodiments, the system is a closed system. In some embodiments, the system is configured to maintain sterility of the substance of interest by avoiding external contaminants. In some embodiments, the system further comprises a pump configured to transfer fluid from the centrifuge unit to the chromatography unit. In some embodiments, the system is configured for continuous processing of the fluid sample. In some embodiments, the system is configured for semi-continuous processing of the fluid sample. In some embodiments, the system is configured for batch processing of the fluid sample. In some embodiments, the fluid sample comprises unlysed cells. In some embodiments, the fluid sample comprises lysed cells. In some embodiments, the fluid sample has a turbidity of from about 0.1 Nephelometric Turbidity Units (NTU) to about 1.0 NTU, from about 1.0 NTU to about 5.0 NTU, from about 5.0 NTU to about 20 NTU, from about 20 NTU to about 50 NTU, from about 50 NTU to about 100 NTU, from about 100 NTU to about 200 NTU, from about 200 NTU to about 300 NTU, from about 300 NTU to about 500 NTU, or greater than about 500 NTU prior to introduction into the system. In some embodiments, the fluid sample has a substance of interest concentration before centrifugation of from about 1 grams/liter (g/L) to about 10 g/L of substance of interest, from about 10 g/L to about 25 g/L, from about 25 g/L to about 30 g/L, from about 30 g/L to about 35 g/L, or greater than 35 g/L.

### DETAILED DESCRIPTION

Disclosed herein are bioprocessing systems 100 (biomolecule collection systems 100) and methods of use thereof that are useful in enriching, purifying, and/or isolating one or more substances of interest (e.g., biomolecules of interest), for example, from a carrier fluid, such as a culture medium or supernatant from a bioprocess container (e.g., a bioreactor).

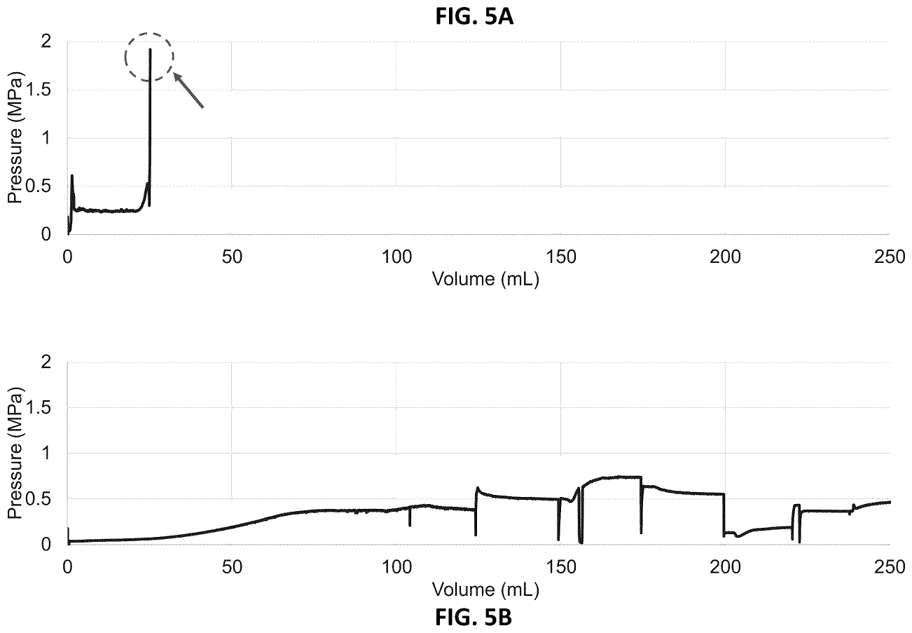

Existing systems and methods for purifying or isolating biomolecules from a carrier fluid suffer from technical shortcomings that are overcome by embodiments of the present disclosure. In particular, existing systems and methods for purifying or isolating biomolecules involve several time-intensive and resource-intensive operational steps. Such steps can include filtration, wherein contaminants and particulates are removed by size exclusion, and affinity chromatography, wherein substances of interest are specifically captured from the carrier fluid and then eluted for collection. The filtration operational step (e.g., depth filters) used in existing systems and methods to remove particulates from the raw input sample (e.g., carrier fluid from a bioprocess container including the biomolecule of interest) can require a substantial and burdensome amount of time to perform. In addition to extending the amount of time required to collect substances of interest from each unit of input sample, the extended duration of the filtration operational step can increase the likelihood of contamination of the sample before the biomolecule of interest is collected, especially when the filtered carrier fluid must be subjected to a product hold step. For example, the product hold step can include a step that technicians perform that is associated with any necessary filter tear-down, column set-up, or fluid transfer steps). Furthermore, filtration devices in such systems can be expensive and need to be replaced regularly. However, existing biomolecule enrichment systems and methods require filtration of sample fluid prior to introduction into the chromatography affinity column. If filtration is not performed in existing systems and methods of biomolecule enrichment before introduction of the sample to the chromatography unit, the chromatography unit quickly becomes clogged with particulates and contaminants, increasing fluid pressure within the system and decreasing processing efficiency until the system fails and comes to a stop (see, FIG. 5A at the circle and arrow, which show a spike in sample pressure and arrest of fluid flow in the system). Accordingly, existing biomolecule enrichment systems and methods are necessarily constrained with respect to the time and cost of biomolecule enrichment and/or isolation by the inclusion of the filtration operational step, which is required for the existing systems and methods to function.

In contrast, systems and methods disclosed herein can be used to enrich, purify, and/or isolate substances of interest (e.g., biomolecules of interest) without a filtration operational step even at large processing volumes, reducing time and cost requirements for production of enriched, purified, or isolated substances of interest. As described herein, the use of a centrifugation step in conjunction with a properly configured affinity chromatography step (for example, without a separate filtration step), can be less expensive, can require less time, and can reduce the risk of contamination during processing as compared to existing systems and methods including a filtration step and any associated product hold step.

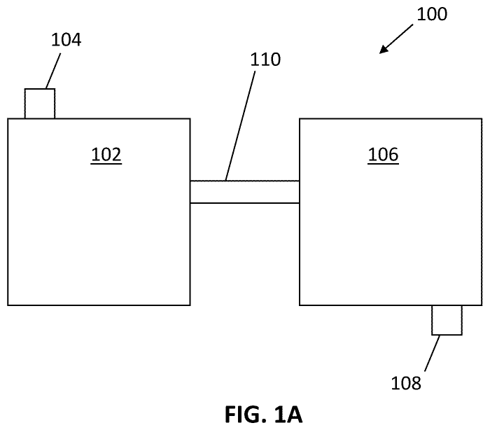

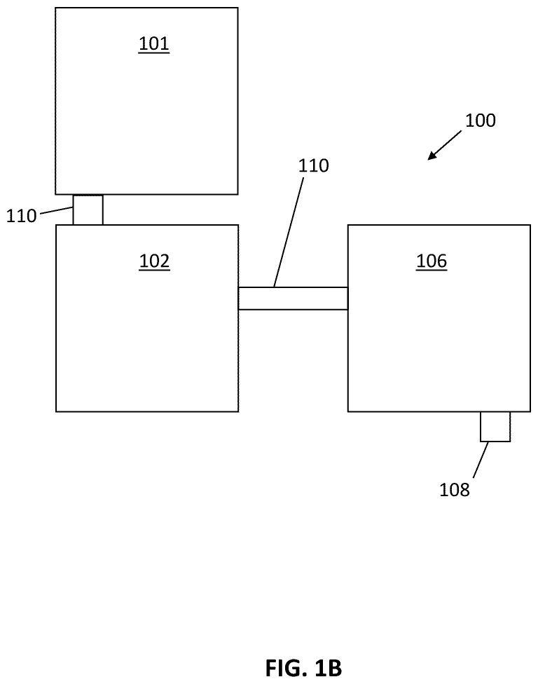

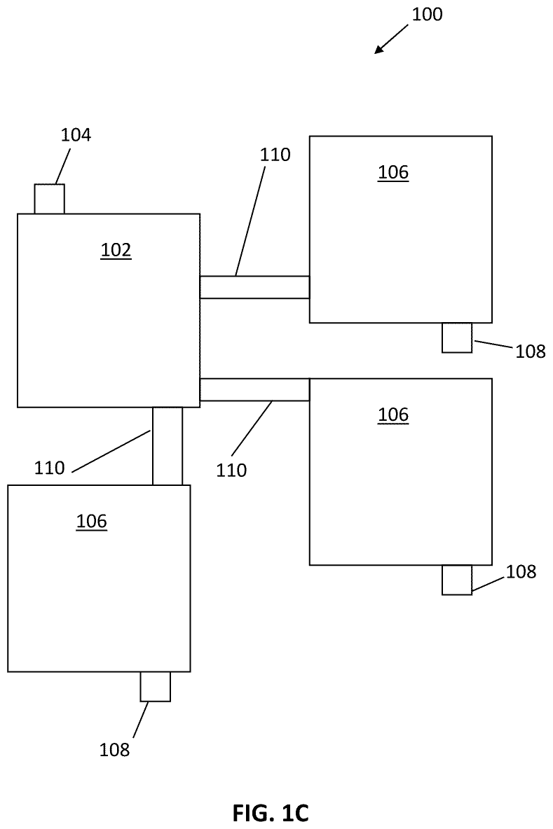

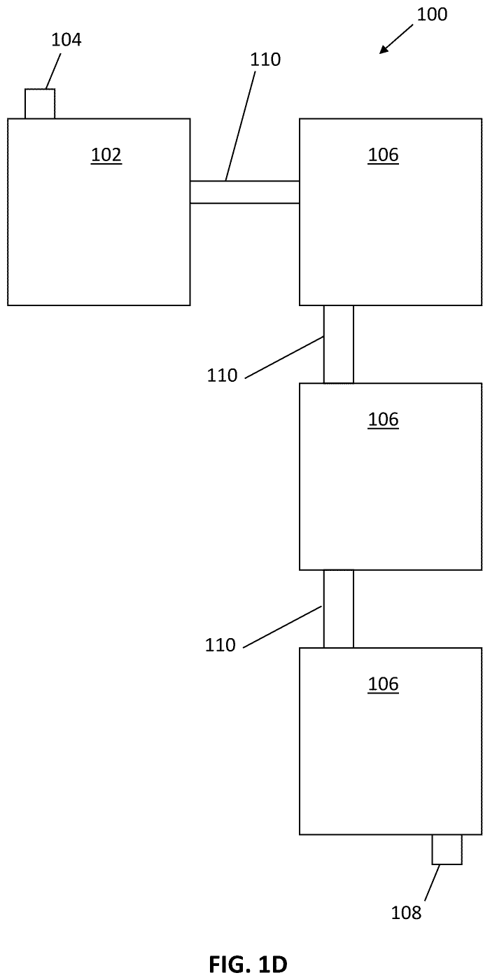

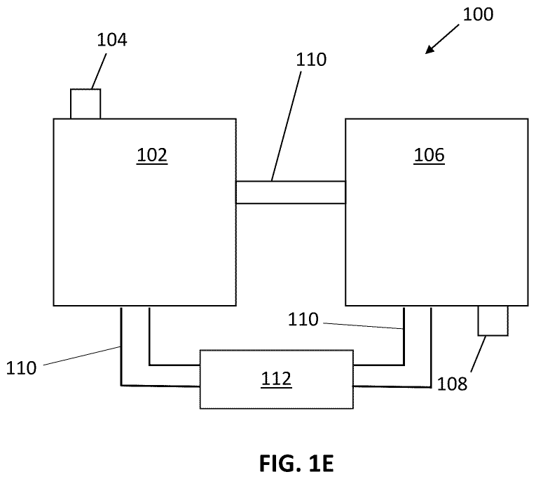

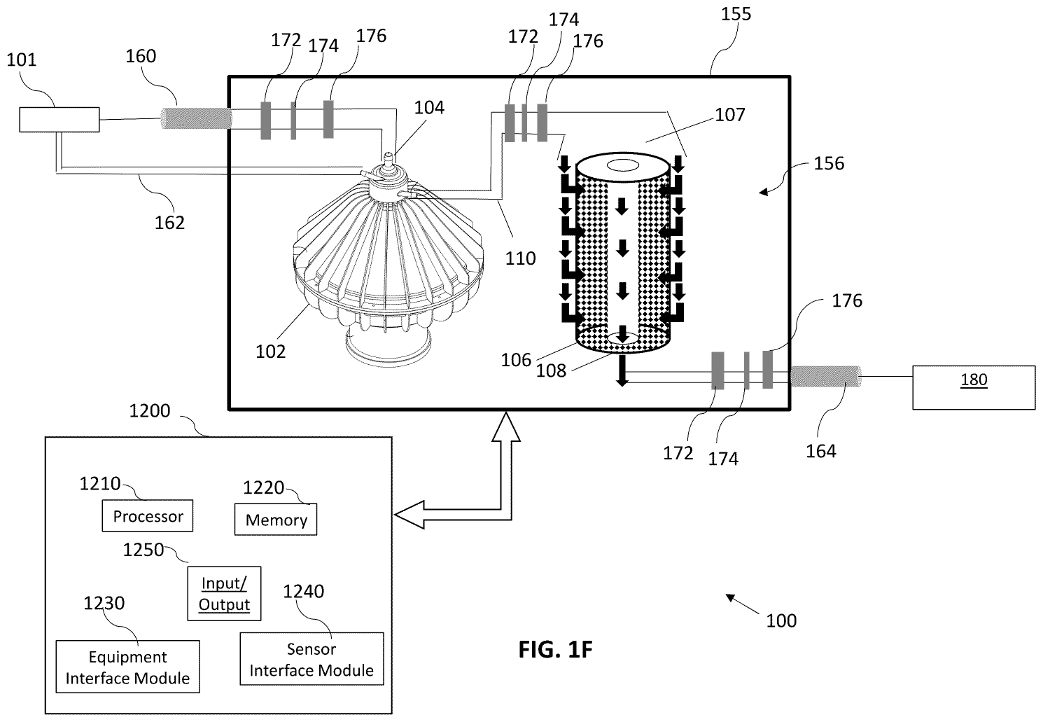

FIGS. 1A-1F show schematics of bioprocessing systems 100 (e.g., biomolecule collection systems 100). Bioprocessing systems 100 (e.g., biomolecule collection systems 100) described herein can include a chromatography unit 106 configured to receive a carrier fluid including a substance of interest (e.g., a molecule of interest, such as a biomolecule of interest) directly from a centrifuge unit 102. A chromatography unit 106 can be configured to capture (e.g., and harvest, for instance by elution) the substance of interest from the carrier fluid (e.g., which may be derived from a sample fluid). In some embodiments, chromatography unit 102 can be a chromatography column (e.g., an affinity chromatography column). The centrifuge unit 102 can be in direct fluid communication with the chromatography unit 106, e.g., by way of one or more fluid pathways 110. In some embodiments, centrifuge unit 102 can be in direct fluid communication with a plurality of chromatography units 106. For example, a centrifuge can be connected to a plurality of chromatography units 106 by a fluidic apparatus comprising a plurality of fluid connections 110. Improved system efficiency can be obtained when each of the one or more fluid pathways provide a direct fluid pathway between the centrifuge unit 102 and a chromatography unit of the one or more chromatography units 106. For instance, improved system efficiency can be obtained in embodiments where the fluid pathway is coupled directly to the centrifuge and directly to the chromatography unit without any intervening processing components (e.g., filters) in fluid communication with the fluid pathway. Accordingly, systems and methods described herein can be used for filter-free harvest and capture of one or more substances of interest (e.g., one or more molecules of interest). As described herein, the combination of pre-processing unclarified samples using centrifugation and use of chromatography units having beads of appropriate material and bead size can allow for continuous, semi-continuous, or batch processing of the samples including a substance of interest (e.g., a biomolecule of interest), thus obviating the need for expensive and time-intensive filtration unit operations prior to chromatography purification.

Centrifuge unit 102 can be used to remove contaminants and/or particulates (e.g., cell debris, microbiota, and/or non-soluble components) from an unclarified fluid sample including the one or more substances of interest (e.g., one or more biomolecules of interest). Centrifugation of an unclarified fluid sample can cause all or a portion of contaminants and/or particulates of an unclarified fluid sample to be pelleted (e.g., physically separated) from the fluid sample (e.g., centrifuged fluid sample, which may be a centrifuge supernatant). Centrifugation of an unclarified fluid sample (e.g., from a bioprocess container 101) can result in a decrease in turbidity of the fluid sample and/or a decrease in the average particle size of the fluid sample (for instance, wherein the centrifuged fluid sample has a lower turbidity and/or a lower average particle size as the fluid sample prior to centrifugation). Centrifugation of a fluid sample (e.g., an unclarified fluid sample) can reduce the concentration and/or average particle size of contaminants and/or particulates compared to initial values for the fluid sample upon introduction into the centrifuge unit 102 (e.g., as transferred from bioprocess container 101). In some embodiments, changes to the fluid sample achieved by centrifugation (e.g., reduction of turbidity, average particle size, and/or concentration of particulates or contaminants) can be critical in preparing the fluid sample for introduction into an chromatography unit 106 (e.g., by directly transferring the fluid sample after centrifugation from the centrifuge unit 102 to the chromatography unit 106). In some embodiments, changes to the fluid sample achieved by centrifugation (e.g., as described herein) are necessary for preparation of the fluid sample such that improvements to throughput and cost efficiency (e.g., by obviation of filtration steps and filtration equipment) can be realized. It is noted that, in some embodiments, centrifugation is not sufficient for realization of improvements to throughput and cost efficiency of biomolecule enrichment, purification, and/or isolation, as described herein. For instance, it can, in some embodiments, be necessary to configure the chromatography unit as described herein so that improvements to throughput and cost efficiency can be realized (e.g., wherein filtration equipment and filtration operational steps are eliminated from the system or method and efficient enrichment, purification, and/or isolation of substance(s) of interest is maintained). In example embodiments, centrifuge unit 102 and associated features and components are disclosed in WO 2022/109612, which is hereby incorporated by reference in its entirety herein.

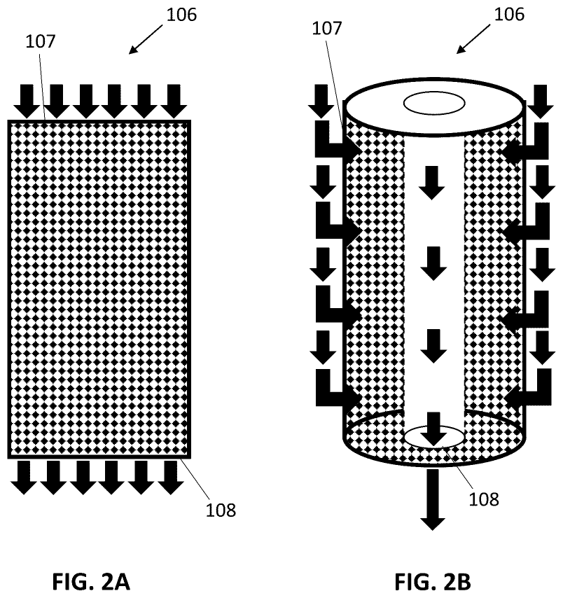

Chromatography unit 106 can include different chromatography columns but not limited to fluidized bed chromatography columns, large-resin chromatography columns (e.g., where resin beads are large and monodisperse in a range of 100 microns to 1000 microns or greater than 1000 microns in diameter), and matrix based chromatographic columns. While the largeness of the resin beads creates relatively large interstitial flow paths for larger contaminants to flow through without clogging in large resin based chromatography columns, matrix-based chromatographic columns, including electro-spun matrices, can be tuned to have properties allowing the flow-through of relatively large contaminants. In example embodiments, chromatography unit 106 can include an affinity chromatography column, including a plurality of beads packed into a housing, wherein all or a portion of the beads include one or more types of affinity tags. In some embodiments, the one or more types of affinity tags can be immobilized on one or more of the beads of a chromatography column (e.g., by chemical attachment) either by conjugating the affinity tag to a molecule directly attached to the bead(s) or by immobilizing the affinity tag directly on the bead(s). An affinity tag can be a molecule (e.g., a protein, nucleic acid, or carbohydrate) capable of specifically binding to (and, in some embodiments, capturing) a substance of interest, such as a biomolecule of interest, or class of substances of interest. In some embodiments, chromatography unit 106 can be configured to pass a fluid (e.g., a fluid sample including a liquid carrier and a substance of interest) into column input 107 and through a matrix of the beads (checkered region of FIG. 2A and FIG. 2B), allowing the affinity tag(s) of the beads to interact with the substances of interest in the fluid (e.g., as shown in FIGS. 2A and 2B). Physical interaction (e.g., contact) of the affinity tag(s) with one or more substances of interest in the fluid (e.g., soluble molecules of interest, such as biomolecules of interest) can result in capture (e.g., mutual association, binding, coupling, or bonding) of the substance(s) of interest by the affinity tag(s). After capture of the substance(s) of interest by the affinity tag(s) of the chromatography column beads, other components of the fluid (e.g., liquid carrier, debris, particulates, and soluble molecules that are not of interest) are allowed to pass through and out of the column (e.g., via column outlet 108). In some embodiments, the pass-through liquid can be transferred (e.g., directly, via a fluidic apparatus of the system 100) to a subsequent chromatography unit for capture of one or more additional substances of interest. The substance(s) of interest captured by (e.g., bound to or associated with) the affinity tag(s) of the chromatography column beads can optionally be washed to further remove residual contaminants, debris, and/or molecules that are not of interest from the column. The substance(s) of interest can then be released (e.g., eluted) into an eluent and collected from the chromatography unit 106 (e.g., chromatography column) by outlet 108, e.g., using a collection container in fluid communication with outlet 108. After collection of the substance(s) of interest (e.g., molecule(s) of interest) in the eluent following affinity chromatography, subsequent unit operations can be performed, including one or more of sterile filtration, pooling of substance(s) of interest, and/or an additional unit operation, such as lyophilization, reconstitution, or chemical modification of the collected substance(s) of interest. In some embodiments, the eluent can be a solvent of a sufficiently high or sufficiently low pH so as to decrease the binding affinity kinetics between the affinity tag and the substance of interest, resulting in the release of the substance of interest into the eluent. For example, an eluent can have a pH of from 1.0 to 2.0, from 2.0 to 3.0, from 3.0 to 4.0, or from 4.0 to 5.0. In some cases, an eluent can be 100 millimolar (mM) acetic acid, 1 molar (M) sodium chloride (NaCl) in water, with a pH of about 2.0. In some embodiments, the eluent is free of contaminants or debris, resulting in a purified sample of the substance of interest. In some embodiments, the volume of the eluent is substantially smaller than the volume of sample fluid passed through the column during capture, resulting in an eluted sample having a much higher concentration of the substance of interest, which can be beneficial for stability of the substance and/or for any subsequent processing steps in the production of a product including the substance of interest. In some embodiments, a concentration of the substance of interest in an eluent at the outlet of the chromatography column can be at least 50% greater, from 50% to 100% greater, from 100% to 150% greater, from 150% to 200% greater, from 200% to 250% greater, or more than 250% greater than a concentration of the substance of interest in the fluid sample at the inlet of the centrifuge. In example embodiments, chromatography unit 106 and associated features and components are disclosed in WO 2022/126115, which is hereby incorporated by reference in its entirety herein.

In some embodiments, it can be necessary to configure the parameters of the chromatography unit 106 appropriately if functionality and efficiency of the system 100 are to be maintained. For example, careful selection of the size and composition of the beads of a chromatography column 106 can be important so as to avoid clogging and/or poor purification efficiency in the system 100. If the average diameter of the beads of the chromatography column is too small, contaminants and debris can begin to clog the column, which can reduce throughput, increase pressure within the column, and/or increase the likelihood of breakthrough of contaminants into the eluent. If the average diameter of the beads of the chromatography column is too large, the capture efficiency (and overall yield) of molecules of interest can be adversely affected. For example, the space between beads can increase with bead diameter, which can result in sample fluid passing between beads without allowing the substance of interest of the sample fluid to come in contact with the affinity tags of the beads. In some cases, providing chromatography column affinity beads of a beneficial average diameter can be insufficient for maintaining throughput and/or cost efficiency benefits of the systems and methods described herein. In some embodiments, it is necessary to perform a centrifugation operational step and to select a beneficial chromatography affinity bead size to obtain certain benefits and advantages of the systems and methods described herein (e.g., wherein the filtration equipment and/or filtration operational step can be eliminated from the system or method).

In some embodiments, the average bead diameter of a population of chromatography column beads can be from 1 micrometer to 500 micrometers, from 1 micrometer to 400 micrometer, from 1 micrometer to 300 micrometers, from 1 micrometer to 200 micrometers, from 1 micrometer to 190 micrometers, from 1 micrometer to 180 micrometers, from 1 micrometer to 170 micrometers, from 1 micrometer to 160 micrometers, from 1 micrometer to 150 micrometers, from 1 micrometer to 100 micrometers, from 1 micrometer to 50 micrometers, from 1 micrometer to 25 micrometers, or from 1 micrometer to 5 micrometers. In some embodiments, the average bead diameter of a population of chromatography column beads can be from 5 micrometers to 500 micrometers, from 5 micrometers to 400 micrometer, from 5 micrometers to 300 micrometers, from 5 micrometers to 200 micrometers, from 5 micrometers to 190 micrometers, from 5 micrometers to 180 micrometers, from 5 micrometers to 170 micrometers, from 5 micrometers to 160 micrometers, from 5 micrometers to 150 micrometers, from 5 micrometers to 100 micrometers, from 5 micrometers to 50 micrometers, or from 5 micrometers to 25 micrometers. In some embodiments, the average bead diameter of a population of chromatography column beads can be from 25 micrometers to 500 micrometers, from 25 micrometers to 400 micrometer, from 25 micrometers to 300 micrometers, from 25 micrometers to 200 micrometers, from 25 micrometers to 190 micrometers, from 25 micrometers to 180 micrometers, from 25 micrometers to 170 micrometers, from 25 micrometer to 160 micrometers, from 25 micrometers to 150 micrometers, from 25 micrometers to 100 micrometers, or from 25 micrometers to 50 micrometers. In some embodiments, the average bead diameter of a population of chromatography column beads can be from 50 micrometers to 500 micrometers, from 50 micrometers to 400 micrometer, from 50 micrometers to 300 micrometers, from 50 micrometers to 200 micrometers, from 50 micrometers to 190 micrometers, from 50 micrometers to 180 micrometers, from 50 micrometers to 170 micrometers, from 50 micrometer to 160 micrometers, from 50 micrometers to 150 micrometers, or from 50 micrometers to 100 micrometers. In some embodiments, the average bead diameter of a population of chromatography column beads can be from 100 micrometers to 500 micrometers, from 100 micrometers to 400 micrometers, from 100 micrometers to 300 micrometers, from 100 micrometers to 200 micrometers, from 100 micrometers to 190 micrometers, from 100 micrometers to 180 micrometers, from 100 micrometers to 170 micrometers, from 100 micrometer to 160 micrometers, or from 100 micrometers to 150 micrometers. In some embodiments, the average bead diameter of a population of chromatography column beads can be from 150 micrometers to 500 micrometers, from 150 micrometers to 400 micrometers, from 150 micrometers to 300 micrometers, or from 150 micrometers to 200 micrometers from 150 micrometers to 190 micrometers, from 150 micrometers to 180 micrometers, from 150 micrometers to 170 micrometers, or from 150 micrometer to 160 micrometers. In some embodiments, the average bead diameter of a population of chromatography column beads can be from 200 micrometers to 500 micrometers, from 20 micrometers to 400 micrometers, or from 200 micrometers to 300 micrometers. In some embodiments, the average bead diameter of a chromatography column beads can be from 300 micrometers to 500 micrometers, from 300 micrometers to 400 micrometers, or from 400 micrometers to 500 micrometers. In particular, systems and methods described herein (e.g., configured for continuous sample processing) can include affinity chromatography beads having an average bead diameter of from 50 micrometers to 100 micrometers or from 100 micrometers to 200 micrometers. In some embodiments, each of the beads of a population of chromatography column beads can be less than 5 micrometers, less than 25 micrometers, less than 50 micrometers, less than 100 micrometers, less than 150 micrometers, less than 160 micrometers, less than 170 micrometers, less than 180 micrometers, less than 190 micrometers, less than 200 micrometers, less than 300 micrometers, less than 400 micrometers, or less than 500 micrometers. In some embodiments, the average bead diameter of a population of chromatography column beads can be less than 5 micrometers, less than 25 micrometers, less than 50 micrometers, less than 100 micrometers, less than 150 micrometers, less than 160 micrometers, less than 170 micrometers, less than 180 micrometers, less than 190 micrometers, less than 200 micrometers, less than 300 micrometers, less than 400 micrometers, or less than 500 micrometers. In some embodiments, the average bead diameter of a population of chromatography column beads can be at least 1 micrometer, at least 5 micrometers, at least 25 micrometers, at least 50 micrometers, at least 100 micrometers, at least 150 micrometers, at least 160 micrometers, at least 170 micrometers, at least 180 micrometers, at least 190 micrometers, at least 200 micrometers, at least 300 micrometers, at least 400 micrometers, or at least 500 micrometers. In some embodiments, the average bead diameter of a population of chromatography column beads can be at most 5 micrometers, at most 25 micrometers, at most 50 micrometers, at most 100 micrometers, at most 150 micrometers, at most 160 micrometers, at most 170 micrometers, at most 180 micrometers, at most 190 micrometers, at most 200 micrometers, at most 300 micrometers, at most 400 micrometers, or at most 500 micrometers.

In some embodiments, at least 75% of a chromatography column's beads can be from 1 micrometer to 500 micrometers, from 1 micrometer to 400 micrometer, from 1 micrometer to 300 micrometers, from 1 micrometer to 200 micrometers, from 1 micrometer to 190 micrometers, from 1 micrometer to 180 micrometers, from 1 micrometer to 170 micrometers, from 1 micrometer to 160 micrometers, from 1 micrometer to 150 micrometers, from 1 micrometer to 100 micrometers, from 1 micrometer to 50 micrometers, from 1 micrometer to 25 micrometers, or from 1 micrometer to 5 micrometers. In some embodiments, at least 75% of a chromatography column's beads can be from 5 micrometers to 500 micrometers, from 5 micrometers to 400 micrometer, from 5 micrometers to 300 micrometers, from 5 micrometers to 200 micrometers, from 5 micrometers to 190 micrometers, from 5 micrometers to 180 micrometers, from 5 micrometers to 170 micrometers, from 5 micrometers to 160 micrometers, from 5 micrometers to 150 micrometers, from 5 micrometers to 100 micrometers, from 5 micrometers to 50 micrometers, or from 5 micrometers to 25 micrometers. In some embodiments, at least 75% of a chromatography column's beads can be from 25 micrometers to 500 micrometers, from 25 micrometers to 400 micrometer, from 25 micrometers to 300 micrometers, from 25 micrometers to 200 micrometers, from 25 micrometers to 190 micrometers, from 25 micrometers to 180 micrometers, from 25 micrometers to 170 micrometers, from 25 micrometer to 160 micrometers, from 25 micrometers to 150 micrometers, from 25 micrometers to 100 micrometers, or from 25 micrometers to 50 micrometers. In some embodiments, at least 75% of a chromatography column's beads can be from 50 micrometers to 500 micrometers, from 50 micrometers to 400 micrometer, from 50 micrometers to 300 micrometers, from 50 micrometers to 200 micrometers, from 50 micrometers to 190 micrometers, from 50 micrometers to 180 micrometers, from 50 micrometers to 170 micrometers, from 50 micrometer to 160 micrometers, from 50 micrometers to 150 micrometers, or from 50 micrometers to 100 micrometers. In some embodiments, at least 75% of a chromatography column's beads can be from 100 micrometers to 500 micrometers, from 100 micrometers to 400 micrometer, from 100 micrometers to 300 micrometers, from 100 micrometers to 200 micrometers, from 100 micrometers to 190 micrometers, from 100 micrometers to 180 micrometers, from 100 micrometers to 170 micrometers, from 100 micrometer to 160 micrometers, or from 100 micrometers to 150 micrometers. In some embodiments, at least 75% of a chromatography column's beads can be from 150 micrometers to 500 micrometers, from 150 micrometers to 400 micrometer, from 150 micrometers to 300 micrometers, or from 150 micrometers to 200 micrometers from 150 micrometers to 190 micrometers, from 150 micrometers to 180 micrometers, from 150 micrometers to 170 micrometers, or from 150 micrometer to 160 micrometers. In some embodiments, at least 75% of a chromatography column's beads can be from 200 micrometers to 500 micrometers, from 20 micrometers to 400 micrometer, or from 200 micrometers to 300 micrometers. In some embodiments, at least 75% of a chromatography column's beads can be from 300 micrometers to 500 micrometers, from 300 micrometers to 400 micrometer, or from 400 micrometers to 500 micrometers. In particular, systems and methods described herein (e.g., configured for continuous sample processing) can include affinity chromatography beads wherein at least 75% of a chromatography column's beads have a diameter of from 50 micrometers to 100 micrometers or from 100 micrometers to 200 micrometers. In some embodiments, at least 75% of a chromatography column's beads can be at least 1 micrometer, at least 5 micrometers, at least 25 micrometers, at least 50 micrometers, at least 100 micrometers, at least 150 micrometers, at least 160 micrometers, at least 170 micrometers, at least 180 micrometers, at least 190 micrometers, at least 200 micrometers, at least 300 micrometers, at least 400 micrometers, or at least 500 micrometers. In some embodiments, at least 75% of a chromatography column's beads can be at most 5 micrometers, at most 25 micrometers, at most 50 micrometers, at most 100 micrometers, at most 150 micrometers, at most 160 micrometers, at most 170 micrometers, at most 180 micrometers, at most 190 micrometers, at most 200 micrometers, at most 300 micrometers, at most 400 micrometers, or at most 500 micrometers.

In some embodiments, at least 80% of a chromatography column's beads can be from 1 micrometer to 500 micrometers, from 1 micrometer to 400 micrometer, from 1 micrometer to 300 micrometers, from 1 micrometer to 200 micrometers, from 1 micrometer to 190 micrometers, from 1 micrometer to 180 micrometers, from 1 micrometer to 170 micrometers, from 1 micrometer to 160 micrometers, from 1 micrometer to 150 micrometers, from 1 micrometer to 100 micrometers, from 1 micrometer to 50 micrometers, from 1 micrometer to 25 micrometers, or from 1 micrometer to 5 micrometers. In some embodiments, at least 80% of a chromatography column's beads can be from 5 micrometers to 500 micrometers, from 5 micrometers to 400 micrometer, from 5 micrometers to 300 micrometers, from 5 micrometers to 200 micrometers, from 5 micrometers to 190 micrometers, from 5 micrometers to 180 micrometers, from 5 micrometers to 170 micrometers, from 5 micrometers to 160 micrometers, from 5 micrometers to 150 micrometers, from 5 micrometers to 100 micrometers, from 5 micrometers to 50 micrometers, or from 5 micrometers to 25 micrometers. In some embodiments, at least 80% of a chromatography column's beads can be from 25 micrometers to 500 micrometers, from 25 micrometers to 400 micrometer, from 25 micrometers to 300 micrometers, from 25 micrometers to 200 micrometers, from 25 micrometers to 190 micrometers, from 25 micrometers to 180 micrometers, from 25 micrometers to 170 micrometers, from 25 micrometer to 160 micrometers, from 25 micrometers to 150 micrometers, from 25 micrometers to 100 micrometers, or from 25 micrometers to 50 micrometers. In some embodiments, at least 80% of a chromatography column's beads can be from 50 micrometers to 500 micrometers, from 50 micrometers to 400 micrometer, from 50 micrometers to 300 micrometers, from 50 micrometers to 200 micrometers, from 50 micrometers to 190 micrometers, from 50 micrometers to 180 micrometers, from 50 micrometers to 170 micrometers, from 50 micrometer to 160 micrometers, from 50 micrometers to 150 micrometers, or from 50 micrometers to 100 micrometers. In some embodiments, at least 80% of a chromatography column's beads can be from 100 micrometers to 500 micrometers, from 100 micrometers to 400 micrometer, from 100 micrometers to 300 micrometers, from 100 micrometers to 200 micrometers, from 100 micrometers to 190 micrometers, from 100 micrometers to 180 micrometers, from 100 micrometers to 170 micrometers, from 100 micrometer to 160 micrometers, or from 100 micrometers to 150 micrometers. In some embodiments, at least 80% of a chromatography column's beads can be from 150 micrometers to 500 micrometers, from 150 micrometers to 400 micrometer, from 150 micrometers to 300 micrometers, or from 150 micrometers to 200 micrometers from 150 micrometers to 190 micrometers, from 150 micrometers to 180 micrometers, from 150 micrometers to 170 micrometers, or from 150 micrometer to 160 micrometers. In some embodiments, at least 80% of a chromatography column's beads can be from 200 micrometers to 500 micrometers, from 20 micrometers to 400 micrometer, or from 200 micrometers to 300 micrometers. In some embodiments, at least 80% of a chromatography column's beads can be from 300 micrometers to 500 micrometers, from 300 micrometers to 400 micrometer, or from 400 micrometers to 500 micrometers. In particular, systems and methods described herein (e.g., configured for continuous sample processing) can comprise affinity chromatography beads wherein at least 80% of a chromatography column's beads have a diameter of from 50 micrometers to 100 micrometers or from 100 micrometers to 200 micrometers. In some embodiments, at least 80% of a chromatography column's beads can be at least 1 micrometer, at least 5 micrometers, at least 25 micrometers, at least 50 micrometers, at least 100 micrometers, at least 150 micrometers, at least 160 micrometers, at least 170 micrometers, at least 180 micrometers, at least 190 micrometers, at least 200 micrometers, at least 300 micrometers, at least 400 micrometers, or at least 500 micrometers. In some embodiments, at least 80% of a chromatography column's beads can be at most 5 micrometers, at most 25 micrometers, at most 50 micrometers, at most 100 micrometers, at most 150 micrometers, at most 160 micrometers, at most 170 micrometers, at most 180 micrometers, at most 190 micrometers, at most 200 micrometers, at most 300 micrometers, at most 400 micrometers, or at most 500 micrometers.

In some embodiments, at least 85% of a chromatography column's beads can be from 1 micrometer to 500 micrometers, from 1 micrometer to 400 micrometer, from 1 micrometer to 300 micrometers, from 1 micrometer to 200 micrometers, from 1 micrometer to 190 micrometers, from 1 micrometer to 180 micrometers, from 1 micrometer to 170 micrometers, from 1 micrometer to 160 micrometers, from 1 micrometer to 150 micrometers, from 1 micrometer to 100 micrometers, from 1 micrometer to 50 micrometers, from 1 micrometer to 25 micrometers, or from 1 micrometer to 5 micrometers. In some embodiments, at least 85% of a chromatography column's beads can be from 5 micrometers to 500 micrometers, from 5 micrometers to 400 micrometer, from 5 micrometers to 300 micrometers, from 5 micrometers to 200 micrometers, from 5 micrometers to 190 micrometers, from 5 micrometers to 180 micrometers, from 5 micrometers to 170 micrometers, from 5 micrometers to 160 micrometers, from 5 micrometers to 150 micrometers, from 5 micrometers to 100 micrometers, from 5 micrometers to 50 micrometers, or from 5 micrometers to 25 micrometers. In some embodiments, at least 85% of a chromatography column's beads can be from 25 micrometers to 500 micrometers, from 25 micrometers to 400 micrometer, from 25 micrometers to 300 micrometers, from 25 micrometers to 200 micrometers, from 25 micrometers to 190 micrometers, from 25 micrometers to 180 micrometers, from 25 micrometers to 170 micrometers, from 25 micrometer to 160 micrometers, from 25 micrometers to 150 micrometers, from 25 micrometers to 100 micrometers, or from 25 micrometers to 50 micrometers. In some embodiments, at least 85% of a chromatography column's beads can be from 50 micrometers to 500 micrometers, from 50 micrometers to 400 micrometer, from 50 micrometers to 300 micrometers, from 50 micrometers to 200 micrometers, from 50 micrometers to 190 micrometers, from 50 micrometers to 180 micrometers, from 50 micrometers to 170 micrometers, from 50 micrometer to 160 micrometers, from 50 micrometers to 150 micrometers, or from 50 micrometers to 100 micrometers. In some embodiments, at least 85% of a chromatography column's beads can be from 100 micrometers to 500 micrometers, from 100 micrometers to 400 micrometer, from 100 micrometers to 300 micrometers, from 100 micrometers to 200 micrometers, from 100 micrometers to 190 micrometers, from 100 micrometers to 180 micrometers, from 100 micrometers to 170 micrometers, from 100 micrometer to 160 micrometers, or from 100 micrometers to 150 micrometers. In some embodiments, at least 85% of a chromatography column's beads can be from 150 micrometers to 500 micrometers, from 150 micrometers to 400 micrometer, from 150 micrometers to 300 micrometers, or from 150 micrometers to 200 micrometers from 150 micrometers to 190 micrometers, from 150 micrometers to 180 micrometers, from 150 micrometers to 170 micrometers, or from 150 micrometer to 160 micrometers. In some embodiments, at least 85% of a chromatography column's beads can be from 200 micrometers to 500 micrometers, from 20 micrometers to 400 micrometer, or from 200 micrometers to 300 micrometers. In some embodiments, at least 85% of a chromatography column's beads can be from 300 micrometers to 500 micrometers, from 300 micrometers to 400 micrometer, or from 400 micrometers to 500 micrometers. In particular, systems and methods described herein (e.g., configured for continuous sample processing) can include affinity chromatography beads wherein at least 85% of a chromatography column's beads have a diameter of from 50 micrometers to 100 micrometers or from 100 micrometers to 200 micrometers. In some embodiments, at least 85% of a chromatography column's beads can be at least 1 micrometer, at least 5 micrometers, at least 25 micrometers, at least 50 micrometers, at least 100 micrometers, at least 150 micrometers, at least 160 micrometers, at least 170 micrometers, at least 180 micrometers, at least 190 micrometers, at least 200 micrometers, at least 300 micrometers, at least 400 micrometers, or at least 500 micrometers. In some embodiments, at least 85% of a chromatography column's beads can be at most 5 micrometers, at most 25 micrometers, at most 50 micrometers, at most 100 micrometers, at most 150 micrometers, at most 160 micrometers, at most 170 micrometers, at most 180 micrometers, at most 190 micrometers, at most 200 micrometers, at most 300 micrometers, at most 400 micrometers, or at most 500 micrometers. In some embodiments, the beads of a chromatography column are polydisperse.

In some embodiments, the beads of a chromatography column are monodisperse. In some cases, a monodisperse population of beads can be advantageous for maintaining consistent percent recovery and breakthrough properties throughout the height (and/or length or width) of the bead bed of the chromatography column. In some embodiments, one or more of the beads of a chromatography column can include a crystalline, semi-crystalline, or amorphous material. In some embodiments, one or more of the beads (e.g., at least 75%, at least 80%, at least 85%, at least 90%, at least 95%, at least 99%, or 100%) of a chromatography column can hold its shape and/or size under fluid flow forces (e.g., forces imparted by at least 1, at least 5, at least 10, at least 15 column volumes per minute). In some embodiments, one or more of the beads of a chromatography column can include a polymer, a glass, a controlled pore glass, an aluminate, a metal, a silicate, encapsulated silica, encapsulated iron particles, encapsulated controlled pore glass, a combination thereof, or a derivative thereof. For instance, one or more of the beads of the chromatography column can include a material selected from a polystyrene, a poly (ethyl) styrene, a polyethylene, a polypropylene, a polyacrylate, a polysaccharide, a native polysilicate, a bonded polysilicate, or a combination or derivative thereof.

In some embodiments, a bead of a chromatography column can include one or more pores. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99%, or 100% of the beads of a chromatography column can include one or more pores. In some embodiments, each pore of the beads can have a diameter of less than 100 angstroms, from about 100 angstroms to about 10,000 angstroms, from about 100 angstroms to about 7,500 angstroms, from about 100 angstroms to about 5,000 angstroms, from about 100 angstroms to about 2,000 angstroms, from about 100 angstroms to about 1,000 angstroms, from about 100 angstroms to about 500 angstroms, from about 100 angstroms to about 250 angstroms, from about 100 angstroms to about 10,000 angstroms, from about 250 angstroms to about 7,500 angstroms, from about 250 angstroms to about 5,000 angstroms, from about 250 angstroms to about 2,000 angstroms, from about 250 angstroms to about 1,000 angstroms, from about 250 angstroms to about 500 angstroms, from about 500 angstroms to about 10,000 angstroms, from about 500 angstroms to about 5,000 angstroms, from about 500 angstroms to about 5,000 angstroms, from about 500 angstroms to about 2,000 angstroms, or from about 500 angstroms to about 1,000 angstroms.

In some embodiments, a bead of a chromatography column can include one or more channels passing therethrough. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99%, or 100% of the beads of a chromatography column can include one or more channels passing therethrough. In some embodiments, each channel of the beads can have a diameter of less than 100 angstroms, from about 100 angstroms to about 10,000 angstroms, from about 100 angstroms to about 7,500 angstroms, from about 100 angstroms to about 5,000 angstroms, from about 100 angstroms to about 2,000 angstroms, from about 100 angstroms to about 1,000 angstroms, from about 100 angstroms to about 500 angstroms, from about 100 angstroms to about 250 angstroms, from about 100 angstroms to about 10,000 angstroms, from about 250 angstroms to about 7,500 angstroms, from about 250 angstroms to about 5,000 angstroms, from about 250 angstroms to about 2,000 angstroms, from about 250 angstroms to about 1,000 angstroms, from about 250 angstroms to about 500 angstroms, from about 500 angstroms to about 10,000 angstroms, from about 500 angstroms to about 5,000 angstroms, from about 500 angstroms to about 5,000 angstroms, from about 500 angstroms to about 2,000 angstroms, or from about 500 angstroms to about 1,000 angstroms.

Beads of a chromatography unit (e.g., an affinity chromatography column) can be packed in the column's housing to form a matrix or bed of packed beads, wherein the matrix or bed has a porosity determined by the void volume fraction within the column. In some cases, the void volume fraction of the chromatography unit 106 (or matrix or bed of affinity beads) can be from 5% to 10%, from 10% to 20%, from 20% to 30%, from 30% to 40%, from 40% to 50%, from 50% to 60%, from 10% to 50%, from 10% to 40%, from 20% to 40%, from 30% to 40%, or greater than 60%. In some embodiments, a desired void volume fraction (e.g., maximum void volume fraction) can be selected by selecting the size and/or type of bead used in the chromatography unit. For instance, a chromatography unit comprising monodisperse spherical resin beads can have a void volume fraction of 30% to 40%, in some embodiments. In some embodiments, a lower void volume fraction can be achieved by reducing the average diameter of a population of beads used in the chromatography unit (e.g., wherein the population of beads is monodisperse).

In some embodiments, a chromatography unit (e.g., an affinity chromatography column) can have an axial flow configuration (AFC), for example, as shown in FIG. 2A. In some embodiments, an axial flow configuration includes loading a fluid (e.g., a centrifuged fluid sample including a substance of interest) into the column at column input 107 and passing the fluid through a matrix of affinity beads (checkered region of FIG. 2A) including affinity tags specific to the one or more substances of interest of the fluid in a direction (see, e.g., arrows of FIG. 2A) substantially parallel to a central long axis of the column and out of column outlet 108. In some embodiments, axial flow configuration can increase the rate of throughput of the chromatography column 106 (e.g., as compared to a column having a radial flow configuration).

In some embodiments, a chromatography unit can have a radial flow configuration (RFC), for example, as shown in FIG. 2B. In some embodiments, a radial flow configuration can include loading a fluid (e.g., a centrifuged fluid sample including a substance of interest) into the column at column input regions 107 (e.g., along sidewalls 107 of column 106 shown in FIG. 2B). The radial flow configuration can further include passing the fluid through a matrix of affinity beads (checkered region of FIG. 2B) including affinity tags specific to the one or more substances of interest of the fluid in a direction (see, e.g., bent arrows of FIG. 2B) generally, but not strictly, perpendicular to a central long axis of the column and out of column outlet 108. In some embodiments, radial flow configuration can increase the interaction of the substance(s) of interest in the carrier fluid of the centrifuged sample with the affinity tags of the beads of the chromatography column.

In some embodiments, a fluid sample (e.g., an unclarified fluid sample including one or more substances of interest) can be introduced to centrifuge unit 102 through centrifuge input 104. As shown in FIG. 1B, a sample fluid can be introduced to centrifuge unit 102 via a fluid pathway 110, for example, from a bioprocess container 101. In such instances, the fluid pathway 110 creating a fluid connection between bioprocess container 101 and centrifuge unit 102 can be a direct, optionally sealed, connection. In some embodiments, the fluid pathway 110 connecting bioprocess container 101 and centrifuge unit 102 can connect bioprocess container 101 to centrifuge unit 102 via centrifuge input 104.

Bioprocess container 101 can have a sample fluid including one or more substances of interest. A substance of interest can be a molecule to be produced, enriched, purified, and/or isolated, for instance for sale as a product or use as a reagent or analyte (e.g., a diagnostic analyte). The sample fluid of a bioprocess container 101 can be a heterogeneous composition. For example, a plurality of soluble and/or insoluble substances may include the sample fluid in the bioprocess container. In some cases, a sample fluid of a bioprocess container can contain soluble proteins, carbohydrates, and/or nucleic acids that are not of interest or which may be deleterious to an enriched population of the substance of interest (e.g., wherein the contaminating substance(s) have enzymatic activity for the substance of interest). Therefore, it can be advantageous to remove contaminants from the fluid including the substance of interest as quickly as possible. By way of example and not by limitation, sample fluid can include one or more biocomponents, including fluids, solids, mixtures, solutions, and suspensions including, but not limited to, bacteria, fungi, algae, plant cells, animal cells, white blood cells, T-cells, cell media, protozoans, nematodes, plasmids, viral vectors, blood, plasma, organelles, proteins, nucleic acids, lipids, plasmids, carbohydrates, and/or other biological components, and the like. Examples of some common biological components that are grown in sample fluid and bioprocess container 101 include E. coli, yeast, bacillus, and CHO cells. Sample fluid can also comprise cell-therapy cultures and cells and microorganisms that are aerobic or anaerobic and adherent or non-adherent. Different media compositions known in the art can be used to accommodate the specific cells or microorganisms grown and the desired end product.

In some embodiments, bioprocess container 101 can be a bioreactor. Container 101 can be configured for biological reactions, including but not limited to, growing cells or other biological components. In example embodiments, bioreactor 120 can also comprise or be substituted with one or more bioreactors, fermenters, mixers, storage vessels, fluid management systems, cell culture equipment, centrifuges, centrifugal separators, chromatography units, mixers, homogenizers, magnetic processing units, blood separating devices, biocomponent filtering devices, biocomponent agitators or any other device designed for growing, mixing or processing cells and/or other biological components. It is also appreciated that bioprocess container 101 can comprise any conventional type of bioreactor, fermenter, or cell culture devices such as a stirred-tank reactor, rocker-type reactor, paddle mixer reactor, or the like. In some uses, bioprocess container 101 primarily grows and recovers cells for subsequent use (e.g., preparing vaccine materials from the cells themselves). But in many uses, the ultimate purpose of growing cells in bioprocess container 101 is to produce and later recover biological products (such as recombinant proteins, viral vectors, etc.) that are exported from the cells into the growth medium. It is also common to use bioprocess container 101 to grow cells in a master batch to prepare a specific volume, density, concentration, CFU and/or aliquot of cells for subsequent use as an inoculant for multiple subsequent batches of cells grown to recover biological products.

In some embodiments, bioprocess container 101 can include live cell culture(s). Bioprocess container 101 can be used to produce one or more substances of interest (e.g., via chemical reaction or one or more biological processes, such as secretion into the sample fluid or enzymatic modification of a substance present in the sample fluid. In some embodiments, the sample fluid of the bioprocess container can include a culture medium. In some embodiments, the sample fluid may contain insoluble material, such as cellular debris (e.g., resulting from a cellular component of bioprocess container 101, which may be involved in production of the substance of interest). Because insoluble components of the sample fluid (e.g., cellular debris) can cause components of a biomolecule collection system 100 (e.g., chromatography unit 106) to clog, it is beneficial to remove such insoluble components in an efficient way. In some embodiments (e.g., some embodiments where the substance of interest is a molecule of interest as compared to a cell population of interest), cellular components may be treated instead as an insoluble contaminant and can be excluded from collection (e.g., by pelleting during centrifugation).

In some embodiments, a centrifuge unit 102 of system 100 can be connected to a plurality of chromatography units 106, wherein the plurality of chromatography units are connected to the centrifuge unit in a parallel arrangement, for instance as shown in FIG. 1C. Systems 100 and methods of this disclosure employing a plurality of chromatography units 106 in parallel arrangement with the centrifuge unit 102 can improve throughput of the system or method, for instance, as the plurality of chromatography units 106 can be used to process carrier fluid including one or more substances of interest at the same time, effectively increasing the total surface area of chromatography units (e.g., surface area of chromatography beads and/or the number of affinity tags on the beads) exposed to the carrier fluid at the same time. Systems 100 and methods of this disclosure can further improve cost savings over a system or method requiring a filtration operational step by employing a plurality of chromatography units 106 arranged in parallel with the centrifuge unit 102, as each of the parallel fluid pathways 110 between centrifuge unit 102 and a chromatography column 106 does not need separate filter (e.g., a frit, porous disc, or porous barrier used to filter a sample) or separate instance of the filtration operational step. In some cases, beads of a first chromatography column can include affinity tags specific for a first substance of interest (e.g., a first biomolecule of interest) and beads of a second chromatography column (e.g., connected to centrifuge unit 102 in parallel with respect to the first chromatography column) can include affinity tags specific for a second substance of interest (e.g., a second biomolecule of interest). In such configurations, it can be possible to enrich, purify, and/or isolate a plurality of different types of substances of interest (e.g., a plurality of different types of biomolecules of interest) from a sample of interest in one production run.

In some embodiments, a centrifuge unit 102 of system 100 can be connected to a plurality of chromatography units 106 in a series arrangement, for instance, as shown in FIG. 1D. Systems 100 and methods of this disclosure employing a plurality of chromatography units 106 in a series arrangement relative to one another (e.g., wherein column output 108 of a first chromatography column is directly connected to column input 107 of a second, downstream chromatography column) can be advantageous. For example, beads of a first chromatography column can include affinity tags specific for a first substance of interest (e.g., a first biomolecule of interest) and beads of a second chromatography column (e.g., connected to centrifuge unit 102 in series with respect to the first chromatography column) can include affinity tags specific for a second substance of interest (e.g., a second biomolecule of interest). In such configurations, it can be possible to enrich, purify, and/or isolate a plurality of different types of substances of interest (e.g., a plurality of different types of biomolecules of interest) from a sample of interest in one production run. Alternatively or additionally, it can be possible to increase capture efficiency of a single substance of interest in the systems 100 and methods disclosed herein by utilizing a plurality of chromatography columns 106 including beads with affinity tags specific for the same substance of interest, wherein the plurality of columns are arranged in series with respect to one another. For example, exceedingly rare or valuable molecules of interest passing through a first chromatography column 106 without being captured by the first chromatography column's affinity tagged beads can be captured by a downstream second chromatography column including beads having affinity tags specific to the same molecule(s) of interest as the first column.

In some embodiments, a centrifuge unit 102 of system 100 can be connected to a plurality of chromatography units 106 in a combination of series and parallel arrangements. For instance, a centrifuge unit 102 can be directly connected in parallel to a first plurality of chromatography units 106, and one or more of the first plurality of primary chromatography units 106 can be subsequently connected in series (e.g., via one or more fluid pathways 110 an output 108 of the one or more primary chromatography unit) to an input 107 of one or more secondary chromatography units 106. Using a combination of parallel and series connections between centrifuge unit 102 and chromatography columns 106 can benefit from the increased throughput of parallel affinity chromatography processing and the benefits to increased capture efficiency per substance of interest type and the option for collecting multiple different substance of interest using separate chromatography columns afforded by chromatography columns connected in series. A person of skill in the art will appreciate that many combinations and arrangements of chromatography columns 106 connected in parallel and in series with respect to one another and/or with respect to centrifuge unit 102 are possible, and all are contemplated and expressly embodied herein.

Bioprocessing systems 100 disclosed herein can comprise a fluidic apparatus comprising one or more fluid pathways 110 (e.g., fluid connections). Fluid pathways 110 of systems 100 and methods disclosed herein can include one or more tubes or other connections configured to route fluid from a first container to a second container. In some embodiments, one or more of the fluid pathways can include one or more valves 174, one or more pumps 176, and/or other means of regulating (e.g., reducing, increasing, or stopping) fluid flow rate through the fluid pathway. In some embodiments, system 100 can include one or more pumps 176 configured to transfer fluid from a first point in the system (e.g., centrifuge unit 102) to a second point in the system (e.g., input 107 of chromatography column 106).

In some embodiments, biomolecule collection system 100 can be a closed system, e.g., wherein the system is configured to maintain the sterility of the fluid sample. For example, connections between one or more of bioprocess container 101, centrifuge unit 102, centrifuge input 104, fluid pathway(s) 110, chromatography column and/or one or more of the components themselves (e.g., centrifuge unit 102, chromatography unit 106, and/or bioprocess container 101) can be sealed (e.g., constituting an airtight, enclosed system) to the outside environment. Embodiments of the present systems 100 and methods wherein all or a portion of the system and/or its components are closed to the outside environment can decrease the likelihood that fluids or substances will be contaminated by factors from the outside environment.

A biomolecule collection system 100 can comprise a closed system, the closed system comprising centrifuge unit 102 and one or more chromatography units 106. In some embodiments, a biomolecule collection system that comprises a closed system can comprise a housing 155 defining a chamber 156 inside of which centrifuge unit 102 and chromatography unit 106 are disposed (e.g., wherein housing 155 bounds the chamber 156). A closed system comprising a fluidic apparatus configured to directly transfer a fluid carrier comprising a substance of interest from the centrifuge unit 102 to the chromatography unit 106 (e.g., after centrifugation), for filter-free capture (e.g., and harvest) of the substance of interest. FIG. 1F shows a diagram illustrating embodiments of a biomolecule collection system 100 comprising a closed system formed within housing 155 in which centrifuge unit 102 and chromatography unit 106 are disposed. Configuring biomolecule collection system 100 to be a closed system having a housing 155 enclosing centrifuge unit 102 and one or more chromatography units 106 (e.g., one or more affinity chromatography columns 106) can decrease the risk of contamination of the sample and/or substance of interest, in addition to other advantageous technical effects and benefits described herein. Centrifuge unit 102 (e.g., an outlet of centrifuge unit 102) can be in fluid communication with one or more respective inlets of the one or more chromatography units 106 within the housing 155 of biomolecule collection system 100. For example, an outlet of centrifuge unit 102 can be connected in parallel to an inlet 107 of a first chromatography unit 106 and to the inlet(s) of one or more additional chromatography units 106. In some embodiments, an outlet of centrifuge unit 102 can be in direct fluid communication with an inlet of a first chromatography unit 106, and an outlet of the first chromatography unit 106 can be in fluid communication with an inlet of one or more additional chromatography units 106 within the housing 155 of the biomolecule collection system 100.

In some embodiments, an inlet 104 of the centrifuge unit 102 can be connected to an inlet port 160 of the housing 155. For example, inlet port 160 can be connected to an inlet 104 of centrifuge unit 102 by a fluid connection 110 (e.g., comprising a tube, a pipe, or other flow path), such that centrifuge unit inlet 104 is in fluid communication with housing inlet port 160. Housing inlet port 160 can comprise a scalable fluid connection, for example, wherein the housing inlet port 160 can maintain sterility of the closed system (e.g., by reducing a risk of or avoiding introduction of external contaminants, such as external biological contaminants) while allowing passage of materials (e.g., fluid sample comprising a substance of interest). In some embodiments. Such configurations can allow introduction of a sample (e.g., from a bioreactor) directly into centrifuge unit 102 without opening the closed system.

A housing inlet 160 can be in fluid communication with a bioprocess container 101 (e.g., bioreactor). For example, bioprocess container 101 can be coupled to a housing inlet 160 (e.g., via a fluid connection 110). In some embodiments, such configurations can place bioprocess container 101 in fluid communication with centrifuge unit 102 across housing 155 without opening the closed system environment of biomolecule collection system 100. In some embodiments, sample fluid comprising a substance of interest (e.g., a molecule of interest) can be passed from bioreactor 101 through housing inlet 160 and into inlet 104 of centrifuge unit 102 (e.g., via one or more fluid connections 110). After transferring a sample comprising a substance (e.g., molecule) of interest from bioprocess container 101 to centrifuge unit 102 (e.g., via housing inlet 160), the sample can be centrifuged (e.g., a centrifugation unit process can be performed on the sample using the centrifuge unit 102), for example, according to method steps described herein. In some embodiments, transferring a fluid from a bioprocess container 101 to centrifugation unit 102 (e.g., through housing inlet 160) can be performed as a result of commands manually input by a user. In some embodiments, transferring a fluid from a bioprocess container 101 to centrifugation unit 102 (e.g., through housing inlet 160) can be performed according to and under the control of a program implemented by a computer system 1200.

In some embodiments, centrifuge unit 102 can be in fluid communication with bioprocess container 101 via feedback line 162. Feedback line 162 can be a fluid connection 110, as described herein. In some preferred embodiments, feedback line 162 can be a separate fluid connection 110 between centrifuge 102 and bioprocess container 101. In some embodiments, a supernatant fluid or remainder fluid can be flowed from centrifuge unit 102 to bioprocess container 101 via feedback line 162, for instance, after a sample comprising a substance (e.g., molecule) of interest is passed to the centrifuge unit from the bioprocess container and a process comprising a centrifugation unit process is performed on the substance of interest by the centrifuge unit 102. In some embodiments, feedback line 162 can be connected to an outflow from the system that is not connected to bioprocess container 101, for instance, wherein feedback line 162 is connected to a waste container or waste outflow. Biomolecule collection system 100 can be configured to pass a fluid from centrifuge unit 102 across a wall of housing 155 via feedback line 162 to bioprocess container 101 (e.g., without opening the closed system within housing 155). In some embodiments, transferring a fluid from centrifugation unit 102 through feedback line 162 (e.g., to bioprocess container 101 or to a waste container) can be performed as a result of commands manually input by a user. In some embodiments, transferring a fluid comprising one or more molecules of interest from centrifugation unit 102 through feedback line 162 (e.g., to bioprocess container 101 or to a waste container) can be performed according to and under the control of a program implemented by a computer system 1200.

One or more molecules of interest (e.g., suspended or dissolved in a carrier fluid, such as sample fluid, clean buffer medium, or another liquid) can be transferred (e.g., flowed) from centrifuge unit 102 to chromatography unit 106 within a housing 155 of a closed system of biomolecule collection system 100 via a fluid connection 110 (for example, as shown in FIG. 1F) after a centrifugation unit process step. A carrier fluid used to transfer the one or more molecules of interest from centrifuge unit 102 to (e.g., an inlet 107 of) chromatography unit 106 can be the same sample fluid in which the substance of interest was transferred from the bioprocess container 101 to the centrifuge unit 102. In some embodiments, a carrier fluid used to transfer the one or more molecules of interest from centrifuge unit 102 to chromatography unit 106 can be a different fluid than the sample fluid in which the substance of interest was transferred from the bioprocess container 101 to the centrifuge unit 102. A fluid (e.g., a centrifuged fluid sample or fresh carrier fluid that has not been used in bioprocess container 101) comprising one or more molecules of interest can be transferred (e.g., flowed) from centrifuge unit 102 to chromatography unit 106 without filtering the fluid. Transferring (e.g., flowing) a fluid comprising one or more molecules of interest from centrifugation unit 102 to chromatography unit 106 within the housing 155 of a closed system of biomolecule collection system 100 after a centrifugation unit process step can advantageously improve the workflow efficiency of the system and decrease the risk of contamination of collected substance of interest. In some embodiments, transferring a fluid comprising one or more molecules of interest from centrifugation unit 102 to chromatography unit 106 can be performed as a result of commands manually input by a user. In some embodiments, transferring a fluid comprising one or more molecules of interest from centrifugation unit 102 to chromatography unit 106 can be performed according to and under the control of a program implemented by a computer system 1200.

One or more (e.g., unfiltered) fluids comprising a substance of interest can be introduced into chromatography unit 106 (e.g., at chromatography unit input 107) via a fluid connection 110 and processed using the chromatography unit 106 (e.g., performing a chromatography unit process) according to steps and methods described herein. In some embodiments, one or more wash steps, hold steps, and/or elution steps can be performed using the chromatography unit (e.g., according to and/or under the control of a computer system 1200). A fluid flowthrough (e.g., eluent) comprising one or more molecules of interest from a chromatography unit process can be flowed (e.g., eluted) out of the chromatography unit through chromatography unit outlet 108). In some embodiments, the fluid flowthrough (e.g., eluent) comprising one or more molecules of interest can be transferred to a product collection unit 180 via a fluid connection 110. In some embodiments, the fluid flowthrough (e.g., eluent) comprising one or more molecules of interest can be transferred to a product collection unit 180 through housing 155 (e.g., via housing outlet 164). In some cases, the fluid connection 110 can be bifurcated and configured to deliver fluid alternatively to product collection unit 180, to a waste collection container or system, or to a wash fluid tank. In some embodiments, a switching mechanism (e.g., comprising a physical flow diversion paddle or a jet flow diversion system) can be used to selectively direct fluid flow to one or more product collection units 180, the waste collection container or system, or, optionally, to a wash fluid tank. In some embodiments, wash fluid can be directed (e.g., across housing wall 155) to a waste collection container or system and eluent comprising a substance of interest can be directed to the one or more product collection units 180.

In some embodiments, a centrifuged sample (e.g., comprising all or a portion of a supernatant present in centrifuge unit 102 after the fluid sample has undergone a centrifuging process in the centrifuge unit 102) can be transferred from an outlet of the centrifuge unit 102 to an inlet of the chromatography unit 106 (e.g., via fluid pathway 110), for instance without the centrifuged sample being subjected to filtration. In some embodiments, the centrifuged sample can be transferred directly from the outlet of the centrifuge unit 102 to the inlet of the chromatography unit 106, without traveling through an additional fluid pathway 110 (e.g., a tube or pipe or other fluid transfer component), for instance without the centrifuged sample being subjected to filtration.

In some embodiments, a bioprocessing system 100 or method of use thereof comprises one or more hold vessels 112 (e.g., a break tank) configured to receive a centrifuged sample from centrifuge unit 102 (e.g., from an outlet of centrifuge unit 102), for example, as shown in FIG. 1E. A hold vessel 112 can be configured to transfer a centrifuged sample to one or more chromatography units 106 (e.g., via the respective input(s) of the of the one or more chromatography units 106). The hold vessel 112 can be in fluid connection with centrifuge unit 102 (e.g., an outlet of centrifuge unit 102) via a fluid pathway 110 (e.g., a fluid connection 110). In some embodiments, the hold vessel 112 can be in fluid communication with one or more chromatography units 106 (e.g., via the respective inlet(s) of the one or more chromatography units 106) via a fluid pathway (e.g., a fluid connection 110). In some embodiments, a hold vessel 112 can be configured to (e.g., temporarily) hold the centrifuged sample prior to introduction into the one or more chromatography units 106. In some embodiments, a first portion of the centrifuged sample can be transferred to the one or more chromatography units 106 while a second portion is still being transferred from the centrifuge unit 102 into the hold vessel 112. In some embodiments, a hold vessel 112 can be used to change a pressure and/or flow rate of the centrifuged sample prior to introduction into the one or more chromatography units 106. For example, the hold vessel 112 can be configured to fill with centrifuged sample at a first flow rate (or first fluid pressure) and to transfer the centrifuged sample to the one or more chromatography units 106 at a second flow rate (or second fluid pressure). In some embodiments, the fluid pressure or flow rate of the centrifuged sample as it is released from the centrifuge unit 102 can be higher than a preferred fluid pressure or flow rate of the centrifuged sample for introduction into the chromatography unit 106 (e.g., because a large volume of centrifuged sample may be prepared by and/or released from the centrifuge unit 102 as a bolus, rather than the centrifuged sample being prepared and/or released continuously from the centrifuge unit 102). In some embodiments, a chromatography unit 106 may be susceptible to damage, increased degradation, or decreased performance (e.g., increased breakthrough) if input flow rate or fluid pressure is too high. Furthermore, chromatography units 106 having different configurations (e.g., axial flow configuration versus radial flow configuration) or comprising different populations of beads (e.g., populations of beads with a different average diameter distribution and/or a different resin type) may require different input fluid flow rates and/or different input fluid pressures. Therefore, it can be advantageous to configure a hold vessel 112 to receive a centrifuged sample at a first flow rate or first fluid pressure and to transfer the centrifuged sample to one or more chromatography units 106 at a second (e.g., lower) flow rate or second (e.g., lower) fluid pressure. In some embodiments, a second flow rate or fluid pressure used to transfer a centrifuged sample from the hold vessel 112 to a chromatography unit 106 (e.g., wherein the second flow rate or fluid pressure is lower than the flow rate or fluid pressure at which the centrifuged sample was introduced into the hold vessel 112) can be achieved in multiple ways, including: differential fluid pathway diameter into the hold vessel 112 and out of the hold vessel 112, active control of fluid restriction and/or pumping, or configuring the size of the hold vessel 112 relative to the expected volume of centrifuged sample produced by the centrifuge unit 102 per centrifuge unit operation (e.g., such that compression of a gas, such as air, in the hold vessel 112 acts as a means of pressure capacitance of the centrifuged sample during filling of the hold vessel 112). In some embodiments, a hold vessel 112 can have an interior volume that is equal in size to the volume of a centrifuge unit 102 centrifugation chamber. In some embodiments, a hold vessel 112 can have an interior volume that is larger than the volume of a centrifuge unit 102 centrifugation chamber. In some embodiments, a hold vessel 112 can have an interior volume that is smaller than the volume of a centrifuge unit 102 centrifugation chamber. In some embodiments, a hold vessel 112 can have an interior volume that is up to 40%, up to 50%, up to 60%, up to 70%, up to 80%, up to 90%, up to 100%, up to 110%, up to 120%, up to 130%, up to 140%, up to 150%, at least 40%, at least 50%, at least 60%, at least 70%, at least 80%, at least 90%, at least 100%, at least 110%, at least 120%, at least 130%, at least 140%, at least 150%, from 40% to 50%, from 50% to 60%, from 60% to 70%, from 70% to 80%, from 80% to 90%, or from 90% to 100% of the volume of a centrifuge unit 102 centrifugation chamber.

Furthermore, a centrifugation unit process may require less time than a subsequent chromatography unit operation. In some embodiments, it can be advantageous to configure the centrifuge unit 102 to transfer centrifuged sample to a plurality of hold vessels 112, e.g., wherein each hold vessel 112 is in fluid communication with one or more chromatography units 106. For example, it may be advantageous to proceed with multiple centrifuge unit operations (e.g., multiple consecutive centrifuge unit operations performed with the same centrifuge unit 106) to produce a plurality of centrifuged sample aliquots and then transfer all or a portion of each of the centrifuged sample aliquots to a plurality of hold vessels 112, e.g., so that multiple chromatography unit operations can be performed in parallel using a plurality of different chromatography units 106, for instance, in situations wherein the chromatography unit operations performed on the centrifuged sample aliquots proceed more slowly or require more time than the centrifuge unit process used to produce the centrifuged sample aliquots.

In some embodiments, the centrifuged sample may be held in the hold vessel 112 for a brief or extended period of time, e.g., until the chromatography unit 106 is ready to receive the centrifuged sample or until a user desires for the centrifuged sample to be transferred to the chromatography unit 106. In some embodiments, holding the centrifuged sample in the hold vessel 112 can advantageously avoid a situation in which the centrifuged sample is held in the chromatography unit 106 prior to the execution of a chromatography unit process on the centrifuged sample (e.g., which may degrade the chromatography medium or an affinity moiety, such as a capture protein, of the chromatography unit 106).

A bioprocessing system 100 (e.g., an automated filter-free harvest and capture system 100) disclosed herein can comprise a system controller 1200, a fluidic apparatus comprising one or more fluid pathways 110, one or more valves 174, one or more pumps 176, one or more sensors 172. An automated bioprocessing system 100 can comprise a housing comprising a chamber in which a centrifuge unit 102 and a chromatography unit 106 are disposed, the centrifuge unit 102 being in fluid communication with the chromatography unit 106. In some embodiments, the automated bioprocessing system 100 can comprise a first set of instructions stored in the memory of the system controller 1200 for flowing a fluid sample from the bioreactor through the centrifuge unit 102 and the chromatography unit 106 to capture and harvest a substance of interest from the fluid sample.

In some embodiments, a fluid pathway 110 (e.g., fluid connection 110), a centrifugation unit 102, and/or a chromatography unit 106 of a biomolecule collection system described herein can comprise one or more of a sensor 172, a valve 174, a pump 176, or a connection port. In some embodiments sensor 172 can be a chemical sensor (e.g., a pH sensor configured to determining a pH of a fluid in the fluid connection), a light-based sensor (e.g., configured to determining an average particle diameter of particles in a fluid, a turbidity of a fluid, or a concentration of one or more molecules or solutes in a fluid within the fluid connection), a temperature sensor, or mechanical sensor (e.g., configured to determine a flow rate, a pressure, and/or a turbulence of a fluid in the fluid connection). A sensor 172 can be operationally coupled to a computer system 1200 of the biomolecule collection system. A sensor 172 can be place at one or more points along one or more fluid pathways 110 of a bioprocessing system's fluidic apparatus. For instance, one or more sensors 172 can be positioned at housing inlet 160, at a fluid pathway 110 between the housing inlet 160 and the centrifuge unit 102, within the centrifuge unit 102, at a fluid pathway 110 between the centrifuge unit 102 and the chromatography unit 106, in the chromatography unit 106, at a fluid pathway 110 between the chromatography unit 106 and the housing outlet 164, at the housing outlet 164, and/or in a collection container 180. In some embodiments, a sensor 172 can be used to detect (e.g., and to determine, along with a computer system 1200) one or more properties of a substance (e.g., molecule) of interest and/or fluids of a biomolecule collection system or process. A valve 174 can be opened and closed to modify (e.g., allow, disallow, increase, or decrease) fluid flow through a fluid connection. In some embodiments, a valve 174 can be controlled (e.g., opened or closed) by a user, for instance, through a physical or digital interface button (e.g., a button on or associated with the system housing) or a mechanical switch, knob, lever, or other means of direct operation of the valve. In some embodiments, a valve 174 can be controlled (e.g., operated) by a computer system 1200 in accordance with user inputs or a program. Operation of one or more valves 174 of a biomolecule collection system can be advantageous in controlling and coordinating steps of a biomolecule collection process, in troubleshooting a malfunction, and/or in adjusting flow parameters to optimize one or more parameters of a process, for instance by increasing, decreasing, interrupting, restoring, or redirecting flow. A pump 176 can be an in-line pump. In some embodiments a pump 176 can be an end-suction pump. In some embodiments, a pump 176 can be a peristaltic pump. Pump(s) 176 of bioprocessing systems 100 (e.g., biomolecule collection systems 100) disclosed herein can be used to convey a fluid carrier or fluid sample along a fluid pathway 110 and/or to transfer a fluid sample or fluid carrier between two system components. A connection port can comprise an adapter configured to interface with a fluid connection, housing, inlet, outlet, bioprocess container, centrifugation unit, or a chromatography unit 106 of a biomolecule collection system. In some embodiments, a connection port can connect opposing ends of two fluid connections 110. In some embodiments, inclusion of one or more connection ports in a biomolecule collection system 100 can allow for easy removal or adjustment of individual components of a biomolecule collection system. For instance, inclusion of connection ports in fluid connections 110 between centrifugation unit 102 and bioprocess container 101 (or housing inlet 160 and/or feedback line 162) and between centrifugation unit 102 and chromatography unit 106 can allow easy removal, maintenance, cleaning, and/or replacement of centrifugation unit 102. Inclusion of connection ports in fluid connections 110 between chromatography unit 106 and housing outlet 164 (or housing inlet 160 and/or feedback line 162) and between centrifugation unit 102 and chromatography unit 106 can allow easy removal (e.g., for a chromatography unit of a different size or configuration), maintenance, cleaning, and/or replacement (e.g., for a fresh chromatography column) of chromatography unit 106.

Bioprocessing systems 100 (e.g., biomolecule collection systems 100) disclosed herein can comprise or be in operational communication with a computer system 1200 (e.g., computer system 1200). Operational control of a computer system 1200 over component(s) of the bioprocessing system 100 can advantageously result in an automated filter-free harvest and capture system. Computer system 1200 comprising a processor and a memory with program instructions stored thereupon that, when executed by the processor, can cause the computer system to operate one or more components of the system 100 and/or to cause one or more process steps disclosed herein to occur using one or more components of the bioprocessing system 100.

A computer system 1200 (e.g., a computer system controller 1200) can include a processor 1210, a memory 1220, an equipment interface module 1230, a sensor interface module 1240, and/or an input/output module 1250. An automated filter-free harvest and capture system (e.g., an automated bioprocessing system 100 disclosed herein) can comprise a computer system controller 1200 comprising a processor 1210 and a memory 1220 (e.g., for storing operational instructions and controlling components of the bioprocessing system). The processor 1210, the memory 1220, the equipment interface module 1230, a sensor interface module 1240, and/or an input/output module 1250 can be interconnected via a system bus. The processor 1210 is capable of processing instructions for execution within the computing system 1200. Such executed instructions can implement one or more components of, for example, a biomolecule collection system 100 or process described herein. In some example embodiments, the processor 1210 can be a single-threaded processor. Alternately, the processor 1210 can be a multi-threaded processor. The processor 1210 is capable of processing instructions stored in the memory 1220 to display graphical information for a user interface provided via the input/output module 1250.

The memory 1220 can be a non-transitory computer-readable medium that stores information within the computing system 1200. The memory 1220 can store data structures representing configuration object databases, for example. The input/output device 1250 provides input/output operations for the computing system 1200. In some example embodiments, the input/output device 1250 can include a physical or virtual keyboard and/or pointing device. In various implementations, the input/output device 1250 can include a display unit for displaying graphical user interfaces. The display unit can be a touch activated screen that displays and facilitates user input/output operations.

According to some example embodiments, the input/output device 1250 can provide input/output operations for a network device. For example, the input/output device 1250 can include Ethernet ports or other networking ports to communicate with one or more wired and/or wireless networks (e.g., a local area network (LAN), a wide area network (WAN), the Internet, a public land mobile network (PLMN), and/or the like). Other communication protocols can include analog, digital and/or other communication signals.

In some example embodiments, the computing system 1200 can be used to execute various interactive computer software applications that can be used for organization, analysis, and/or storage of data in various formats. Alternatively, the computing system 1200 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing items and/or functionalities. Upon activation within the applications, the functionalities can be used to generate the user interface provided via the input/output device 1250. The user interface can be generated and presented to a user by the computing system 1200 (e.g., on a computer screen monitor, etc.).

In some embodiments, a memory 1220 of computing system 1200 (e.g., system controller 1200) can comprise instructions stored thereupon that, when executed by processor 1210 of the computer system 1200, can operate one or more components of a system or device described herein and/or can cause the system or device to perform one or more method steps described herein. Equipment interface module 1230 can be configured to operate one or more components of biomolecule collection system 100. For example, equipment interface module 1230 can provide commands from computer system 1200 to centrifuge unit 102 to perform a centrifugation unit process step or method and/or to chromatography unit 106 to perform a chromatography unit process step or method. In some embodiments, equipment interface module 1230 can be configured to operate pumps, valves, doors, motors, vacuums and/or fluid reservoirs for moving a fluid from a first position to a second position in biomolecule collection system 100. Sensor interface module 1240 can be configured to process data received from one or more sensors 172 of biomolecule collection system 100 (e.g., in embodiments wherein one or more sensors 172 of bioprocessing system 100 is in electronic communication with computer system controller 1200). In some embodiments, sensor interface module 1240 can be configured to determine and/or initialize one or more processes or method steps in response to the processed sensor data (e.g., by comparing the sensor data to a relative, conditional, or absolute threshold for one or more parameters, wherein the threshold may be determined by a user or defined by a program of the computer system 1200.

In some cases, definition of parameters for, initiation of, abortion of, and/or modification of one or more steps of one or more unit processes can be performed manually by a user. In some cases, definition of parameters for, initiation of, abortion of, and/or modification of one or more steps of one or more unit processes can be performed according to and/or under the control of a program implemented by a computer system 1200. For example, operating parameters, initiation of, abortion of, and/or modification of one or more steps in a centrifugation unit process can be performed according to and/or under the control of a program implemented by a computer system 1200. Computer system control of parameters for, initiation of, abortion of, and/or modification of one or more steps in one or more unit processes of a biomolecule collection method and/or used by a biomolecule collection system 100 described herein can allow real-time adjustments of unit processes (e.g., based on sensor data collected at one or more portions of the process and/or one or more locations of the system). Computer system control of parameters for, initiation of, abortion of, and/or modification of one or more steps of one or more unite processes of a biomolecule collection method and/or used by a biomolecule collection system 100 described herein can allow a closed system of the method or system (e.g., as shown in FIG. 1F) to remain closed to the outside environment.

A substance of interest can be a molecule of interest or a complex of molecules of interest (e.g., two or more molecules that are physically associated with one another, for instance through a chemical bond or an impermanent physical association). In some cases, a substance of interest can be a biomolecule. A biomolecule can be a molecule produced by a living cell. A biomolecule can be a protein, a carbohydrate, a nucleic acid (e.g., ribonucleic acid (RNA) or deoxyribonucleic acid (DNA)), or a lipid. In some embodiments, a protein can be an antibody (e.g., a monoclonal antibody). In some embodiments, a protein can be a viral capsid. In some embodiments, a protein can be an enzyme. In some embodiments, a protein can be a structural protein (e.g., elastin or a collagen). In some embodiments, a protein can be a cell signaling protein, such as insulin or a hormone. In some embodiments, a protein can be a cell receptor (e.g., a cell surface receptor). In some embodiments, a nucleic acid can be a cell-free DNA, a complementary DNA (cDNA) or genomic DNA. In some embodiments, a nucleic acid (e.g., an oligonucleotide) can be a synthetic DNA molecule or a synthetic RNA molecule. In some embodiments, a nucleic acid can be a messenger RNA (mRNA), a transfer RNA (tRNA), or a ribosomal RNA (rRNA). In some embodiments, an RNA can be a micro-RNA (miRNA), or a short interfering RNA (siRNA). In some embodiments, a nucleic acid can be single-stranded. In some embodiments, a nucleic acid can be double-stranded. In some cases, a molecule of interest can be a combination of a protein and a nucleic acid (e.g., a protein-nucleic acid complex). In some embodiments, a carbohydrate can include a monosaccharide or a polysaccharide. In some embodiments, a carbohydrate can be a starch. In some embodiments, a lipid can be a fatty acid, a phospholipid, a lipoprotein, a glycolipid, or a steroid. In some embodiments, a sample fluid can include a plurality of molecules of interest.

A bead of a chromatography column can include an affinity tag. In some embodiments, an affinity tag of a bead can include a lipid. In some embodiments, an affinity tag of a bead can include a carbohydrate. In some embodiments, an affinity tag of a bead can include a nucleic acid. In some embodiments, an affinity tag of a bead can include a protein. In some embodiments, an affinity tag of a bead can include a lipoprotein. In some embodiments, an affinity tag of a bead can be protein A. In some embodiments, an affinity tag can include a binding target (e.g., a ligand) of an antibody. In some embodiments, an affinity tag can include a binding target for an adeno-associated virus (AAV). In some embodiments, an affinity tag can include a binding target for an adenovirus. In some embodiments, an affinity tag can include a binding target for a lentivirus. In some embodiments, an affinity tag can include a binding target for a retrovirus.

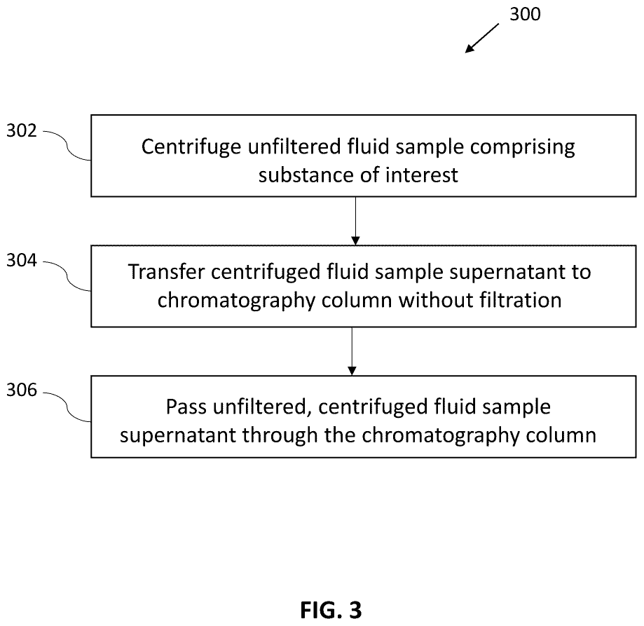

Methods of collecting a substance of interest can include utilizing a system 100, combination of systems 100, or a combination of components of a system disclosed herein. Methods of collecting a substance disclosed herein can reduce the time required to collect (e.g., enrich, purify, or isolate) a substance of interest, reduce the cost to collect the substance of interest, and/or improve the efficiency of purification of a substance of interest. FIG. 3 shows steps of a method 300 of collecting a substance of interest disclosed herein. Step 302 indicates that a method can include centrifuging an unfiltered fluid sample including one or more substance of interest. In some embodiments, the sample input into the centrifuge unit 102 can be the (e.g., unprocessed) fluid sample of a bioprocess container (e.g., during or after a bioprocess, such as a production of a substance into the sample fluid via cell culture). In some embodiments, the sample fluid is unfiltered and/or unclarified when it is introduced into the centrifuge.

In some embodiments, the fluid sample transferred to the centrifuge unit (e.g., from a bioprocess container) has a turbidity of from about 1.0 Nephelometric Turbidity Units (NTU) to about 100 NTU, from about 100 NTU to about 250 NTU, from about 250 NTU to about 500 NTU, from about 500 NTU to about 750 NTU, from about 750 NTU to about 1,000 NTU, from about 1,000 NTU to about 2,500 NTU, from about 2,500 NTU to about 5,000 NTU, from about 5,000 NTU to about 10,000 NTU, or greater than about 10,000 NTU prior to introduction into the system. In some embodiments, the centrifuged sample (e.g., the centrate or supernatant resulting from a centrifuge unit operation) has a turbidity of from about 0.1 Nephelometric Turbidity Units (NTU) to about 1.0 NTU, from about 1.0 NTU to about 5.0 NTU, from about 5.0 NTU to about 20 NTU, from about 20 NTU to about 50 NTU, from about 50 NTU to about 100 NTU, from about 100 NTU to about 200 NTU, from about 200 NTU to about 300 NTU, from about 300 NTU to about 500 NTU, or greater than about 500 NTU after centrifugation. In some embodiments, an eluent collected from the chromatography unit has a turbidity of from about 0.1 Nephelometric Turbidity Units (NTU) to about 0.01 NTU, from about 0.5 NTU to about 1.0 NTU, from about 1.0 NTU to about 2.5 NTU, from about 2.5 NTU to about 5.0 NTU, from about 0.01 NTU to about 5.0 NTU, from about 0.5 NTU to about 5.0 NTU, from about 1.0 NTU to about 5.0 NTU, from about 2.5 NTU to about 5.0 NTU, or greater than about 5.0 NTU. In some embodiments, the fluid sample has concentration of a substance of interest (e.g., before centrifugation) of from about 1 grams/liter (g/L) to about 10 g/L of substance of interest, from about 10 g/L to about 25 g/L, from about 25 g/L to about 30 g/L, from about 30 g/L to about 35 g/L, or greater than 35 g/L. In some embodiments, the fluid sample can be centrifuged at a force of at least 500×g, at least 1,000×g, at least 3,000×g, at least 5,000×g, at least 10,000×g, at least 15,000×g, or at least 30,000×g. For example, a force of from 500×g to 1,000×g (or higher) can be useful in separating (e.g., pelleting) large particulates (e.g., cells) from the fluid sample. A force of from 1,000×g to 10,000×g can be useful in separating cells and cell debris from the fluid sample. A force of greater than 10,000×g can be useful in separating microvesicles and organelles from the fluid sample. In some embodiments, the centrifuged sample fluid (e.g., the unfiltered fluid supernatant of the sample following centrifugation) can have a lower turbidity value than the fluid sample prior to centrifugation. In some embodiments, the centrifuged sample fluid (e.g., the unfiltered fluid supernatant of the sample following centrifugation) can have a lower average particle size than the fluid sample prior to centrifugation.

In some embodiments, the chromatography unit 106 can be constructed from one or more layers (e.g., a plurality of layers) of electrospun textile, for example, such that the interstitial volumes allow for flow of the residual particles after centrifugation. The electrospun fiber diameter can range from 0.1 micrometer to 10 micrometers. The electrospun fibers can be composed of one or more of polystyrene, polyethylene, polypropylene, polysaccharide, cellulose, silica, or agarose materials. The void volumes can be similar to the packed resin void volumes described previously in this disclosure. The electrospun fiber can also be distributed within a supported bed or a microporous membrane format.

In some implementations, performing a centrifugation operation (e.g., as described herein) can result in a centrifuged sample having an average particle diameter of less than 1 micrometer, less than 2 micrometers, less than 3 micrometers, less than 4 micrometers, less than 5 micrometers, less than 6 micrometers, less than 7 micrometers, less than 8 micrometers, less than 9 micrometers, less than 10 micrometers, at least 1 micrometer, at least 2 micrometers, at least 3 micrometers, at least 4 micrometers, at least 5 micrometers, at least 6 micrometers, at least 7 micrometers, at least 8 micrometers, at least 9 micrometers, or at least 10 micrometers. In some embodiments, a centrifuged sample of the systems and methods described herein can be monodisperse. In some embodiments, a centrifuged sample of the systems and methods described herein can be polydisperse. Chromatography units or processes (e.g., as described herein) can be used to process a centrifuged sample having an average particle diameter of less than 1 micrometer, less than 2 micrometers, less than 3 micrometers, less than 4 micrometers, less than 5 micrometers, less than 6 micrometers, less than 7 micrometers, less than 8 micrometers, less than 9 micrometers, less than 10 micrometers, at least 1 micrometer, at least 2 micrometers, at least 3 micrometers, at least 4 micrometers, at least 5 micrometers, at least 6 micrometers, at least 7 micrometers, at least 8 micrometers, at least 9 micrometers, or at least 10 micrometers. For example, a chromatography unit or process described herein comprising an average bead size of up to 50 micrometers can be used to process a centrifuged sample having an average particle size of less than 1 micrometer, less than 2 micrometers, less than 3 micrometers, less than 4 micrometers, less than 5 micrometers, less than 6 micrometers, less than 7 micrometers, less than 8 micrometers, less than 9 micrometers, less than 10 micrometers, at least 1 micrometer, at least 2 micrometers, at least 3 micrometers, at least 4 micrometers, or at least 5 micrometers. In some embodiments, a chromatography unit or process described herein comprising an average bead size of from 50 micrometers to 200 micrometers can be used to process a centrifuged sample having an average particle size of less than 1 micrometer, less than 2 micrometers, less than 3 micrometers, less than 4 micrometers, less than 5 micrometers, less than 6 micrometers, less than 7 micrometers, less than 8 micrometers, less than 9 micrometers, less than 10 micrometers, at least 1 micrometer, at least 2 micrometers, at least 3 micrometers, at least 4 micrometers, or at least 5 micrometers, at least 6 micrometers, at least 7 micrometers, at least 8 micrometers, at least 9 micrometers, or at least 10 micrometers.

A step 304 can include transferring the centrifuged fluid sample (e.g., the unfiltered fluid supernatant of the sample after centrifugation) directly to a chromatography unit. In some embodiments, directly transferring the centrifuged fluid sample from the centrifuge unit to the chromatography unit does not include filtering the centrifuged fluid sample. The centrifuged fluid sample can be transferred to the chromatography unit via fluid pathway 110 (e.g., a fluid pathway that does not include a filter and includes the entirety of the only connection(s) between the centrifuge unit 102 and the chromatography column input 107). In some embodiments, the centrifuged fluid sample does not pass through a filter between the step of centrifuging the unfiltered fluid sample and the step of passing the unfiltered, centrifuged fluid sample supernatant including the substance of interest through the chromatography column (e.g., wherein the column includes a plurality of beads having affinity tags configured to specifically capture the substance of interest).

A step 306 can include passing the centrifuged fluid sample (e.g., the unfiltered fluid supernatant of the sample after centrifugation) through the chromatography unit (e.g., contacting the affinity tags of beads of the chromatography column with the substance of interest of the fluid sample to cause capture of the substance of interest by the affinity tags).

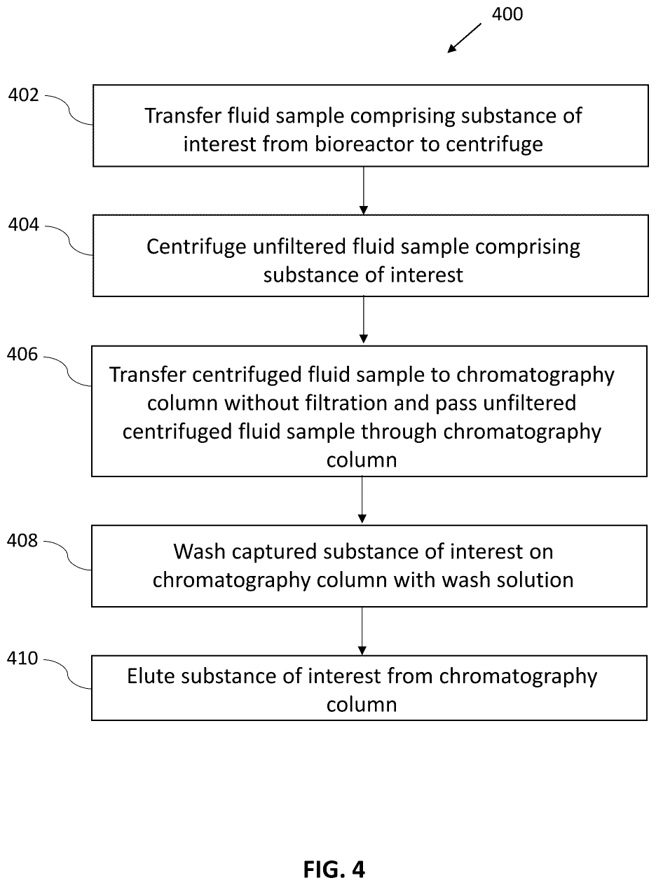

Steps of a method 400 for collecting substances of interest from a fluid sample are shown in FIG. 4. Method 400 can include a step 402, wherein all or a portion of a fluid sample including a substance of interest is transferred from a bioprocess container (e.g., a bioreactor) to a centrifuge unit (e.g., without subjecting the fluid sample to a filtration operational step. Method 300 can include a step 404, wherein the unfiltered fluid sample including the substance of interest is centrifuge, as described herein. Step 406 of method 400 can include transferring the centrifuged fluid sample to a chromatography unit without filtering the fluid sample. Step 408 of method 400 can include washing the substance of interest captured on the chromatography column with a wash solution. Step 410 of method 400 can include eluting the substance of interest from the chromatography column (e.g., into a collection container) using an eluent.

The selection of bead diameter for use in the chromatography unit can affect performance (e.g., percent recovery, load capacity, and/or volumetric throughput) of a method described herein (e.g., method 300 or method 400) with respect to systems 100. In some embodiments, it can be advantageous to pass the centrifuged fluid sample through a chromatography unit including beads having an average diameter of from 50 micrometers to 100 micrometers or from 100 micrometers to 200 micrometers at step 306 or step 406. For example, it is possible to introduce a centrifuged, unfiltered fluid sample comprising one or more substances of interest directly from the centrifuge unit to the chromatography column without passing the sample fluid through a filter when chromatography beads of an appropriate average diameter are selected. In some implementations, a method 300, a method 400, or a method including one or more steps of method 300 or method 400 can be performed two or more times. In some embodiments, a chromatography column can be replaced after use in a method 300, a method 400, or a method including one or more steps of method 300 or method 400. In some embodiments, a chromatography column can be reused in a method 300, a method 400, or a method including one or more steps of method 300 or method 400 after 1, 2, 3, 4, 5, 6, 7, 8, 9, 10, or more than 10 uses.

FIG. 5A shows pressure data illustrating the insufficiency of introducing unfiltered fluid sample directly into a chromatography column without centrifugation, and FIG. 5B show pressure data for an identical experimental design, wherein the fluid sample is subjected to centrifugation as described herein and introduced into the chromatography column without filtering the fluid sample. The data presented in FIGS. 5A and 5B were produced using a 5 milliliter (mL) pre-packed axial flow column (AFC), with polystyrene beads having an average diameter of 50 micrometers. The target molecule for separation was introduced at a concentration of 1.94 mg/mL in an unfiltered fluid containing 49.6×10⁶ cells/mL. After centrifugation, the centrifuged sample contained 0.44×10⁶ cells/mL, and the centrifuged sample was introduced into the chromatography unit without filtration at a rate of 1.05 mL/min (125 cm/hour). As can be seen in FIG. 5A, the pressure (shown in megapascals, MPa) of the uncentrifuged fluid sample rises rapidly upon introduction of the fluid sample into the chromatography unit, resulting in system failure and unscheduled truncation of the experiment (circle and arrow). In contrast, FIG. 5B shows that processing of fluid samples in an identical chromatography unit proceeds indefinitely (experimental data was collected until more than 100 mL of fluid sample were processed) when the fluid sample has been centrifuged and introduced directly into the chromatography column without filtration, as described herein. One advantage of the systems and methods disclosed herein illustrated by this data is that the systems and methods disclosed herein can be used to process fluid sample (e.g., centrifuged, unfiltered fluid sample) continuously in addition to semi-continuously or in discrete batches. For example, the systems and methods disclosed herein can allow for continuous processing and collection of substance(s) of interest from a fluid sample. For instance, some embodiments of systems and methods disclosed herein can include a continuous processing configuration, e.g., wherein the fluid sample is transferred from the centrifuge unit to one or more chromatography columns (for example, two or more chromatography columns in parallel fluid communication with the centrifuge) such that no pause in collection (e.g., to allow for washing of the chromatography column) is needed.

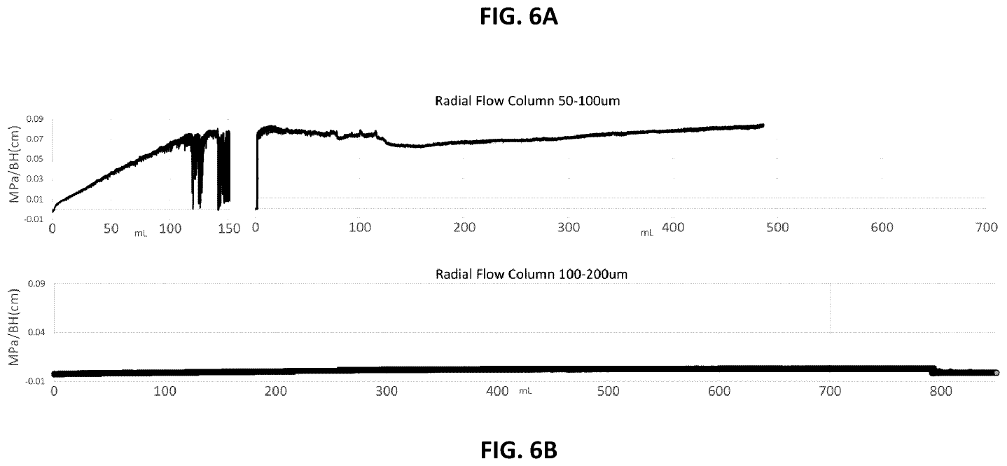

FIG. 6A shows chromatography unit pressure data collected in experiments using a chromatography unit configured in a radial flow configuration and loaded with beads having an average diameter of from 50 micrometers (μm) to 100 micrometers. The data shows that at least about 600 mL of centrifuged, unfiltered fluid sample (wherein the fluid sample includes lysed cells, resulting in substantial intercellular contaminants in the sample) can be processed through the chromatography unit when systems and methods described herein are used. In the experiment reflected in the data of FIG. 6, an air bubble in the line caused a brief disruption in processing after approximately 100 milliliters (mL) had been processed through the system; however, the air bubble was cleared, and the system was used to process at least an additional 475 mL of centrifuged, unfiltered sample (at least 575 mL total) without excessive pressure buildup in the system (e.g., pressure normalized to chromatography column bed height (MPa/BH(cm)) never rose above 0.09), as shown in FIG. 6. FIG. 6B shows chromatography unit pressure data collected in experiments using a chromatography unit configured in a radial flow configuration and loaded with beads having an average diameter of from 100 micrometers to 200 micrometers. The data shows that centrifuged, unfiltered fluid sample can be processed by the system indefinitely, as over 800 mL of centrifuged, unfiltered fluid sample was processed by the system with the normalized pressure (MPa/BH(cm)) of the system never rising above 0.01. In some embodiments, systems and methods described herein including a chromatography column having beads of average diameter from 50 micrometers to 100 micrometers can process from 1 milliliter (mL) to 10,000 milliliters (mL), from 100 mL to 10,000 mL, from 200 mL to 10,000 mL, from 500 mL to 10,000 mL, from 1,000 mL to 10,000 mL, from 2,000 mL to 10,000 mL, from 5,000 mL to 10,000 mL, from 7,500 mL to 10,000 mL, at least 100 mL, at least 200 mL, at least 300 mL, at least 400 mL, at least 500 mL, at least 600 mL, at least 700 mL, at least 800 mL, at least 900 mL, at least 1,000 mL, at least 2,000 mL, at least 5,000 mL, or greater than 10,000 mL of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 50 micrometers to 100 micrometers can process greater than 1 milliliter (mL), greater than 100 mL, greater than 200 mL, greater than 500 mL, or greater than 1,000 mL, greater than 2,000 mL, greater than 5,000 mL, greater than 7,500 mL, or greater than 10,000 mL of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 50 micrometers to 100 micrometers can process from 1 milliliter (mL) to 10,000 milliliters (mL), from 100 mL to 10,000 mL, from 200 mL to 10,000 mL, from 500 mL to 10,000 mL, from 1,000 mL to 10,000 mL, from 2,000 mL to 10,000 mL, from 5,000 mL to 10,000 mL, from 7,500 mL to 10,000 mL, or greater than 10,000 mL of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) docs not exceed 0.10. In some embodiments, systems and methods described herein including a chromatography column having beads of average diameter from 50 micrometers to 100 micrometers can process from 1 liter (L) to 10,000 liters (L), from 100 L to 10,000 L, from 200 L to 10,000 L, from 500 L to 10,000 L, from 1,000 L to 10,000 L, from 2,000 L to 10,000 L, from 5,000 L to 10,000 L, from 7,500 L to 10,000 L, at least 100 L, at least 200 L, at least 300 L, at least 400 L, at least 500 L, at least 600 L, at least 700 L, at least 800 L, at least 900 L, at least 1,000 L, at least 2,000 L, at least 5,000 L, or greater than 10,000 L of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 50 micrometers to 100 micrometers can process greater than 1 liter (L), greater than 100 L, greater than 200 L, greater than 500 L, or greater than 1,000 L, greater than 2,000 L, greater than 5,000 L, greater than 7,500 L, or greater than 10,000 L of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 50 micrometers to 100 micrometers can process from 1 liter (L) to 10,000 liters (L), from 100 L to 10,000 L, from 200 L to 10,000 L, from 500 L to 10,000 L, from 1,000 L to 10,000 L, from 2,000 L to 10,000 L, from 5,000 L to 10,000 L, from 7,500 L to 10,000 L, or greater than 10,000 L of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 100 micrometers to 200 micrometers can process from 1 milliliter (mL) to 10,000 milliliters (mL), from 100 mL to 10,000 mL, from 200 mL to 10,000 mL, from 500 mL to 10,000 mL, from 1,000 mL to 10,000 mL, from 2,000 mL to 10,000 mL, from 5,000 mL to 10,000 mL, from 7,500 mL to 10,000 mL, at least 100 mL, at least 200 mL, at least 300 mL, at least 400 mL, at least 500 mL, at least 600 mL, at least 700 mL, at least 800 mL, at least 900 mL, at least 1,000 mL, at least 2,000 mL, at least 5,000 mL, or greater than 10,000 mL of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 100 micrometers to 200 micrometers can process greater than 1 milliliter (mL), greater than 100 mL, greater than 200 mL, greater than 500 mL, or greater than 1,000 mL, greater than 2,000 mL, greater than 5,000 mL, greater than 7,500 mL, or greater than 10,000 mL of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 100 micrometers to 200 micrometers can process from 1 milliliter (mL) to 10,000 milliliters (mL), from 100 mL to 10,000 mL, from 200 mL to 10,000 mL, from 500 mL to 10,000 mL, from 1,000 mL to 10,000 mL, from 2,000 mL to 10,000 mL, from 5,000 mL to 10,000 mL, from 7,500 mL to 10,000 mL, or greater than 10,000 L of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 100 micrometers to 200 micrometers can process from 1 liter (L) to 10,000 liters (L), from 100 L to 10,000 L, from 200 L to 10,000 L, from 500 L to 10,000 L, from 1,000 L to 10,000 L, from 2,000 L to 10,000 L, from 5,000 L to 10,000 L, from 7,500 L to 10,000 L, at least 100 L, at least 200 L, at least 300 L, at least 400 L, at least 500 L, at least 600 L, at least 700 L, at least 800 L, at least 900 L, at least 1,000 L, at least 2,000 L, at least 5,000 L, or greater than 10,000 L of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 100 micrometers to 200 micrometers can process from 1 liter (L) to 10,000 milliliters (L), from 100 L to 10,000 L, from 200 L to 10,000 L, from 500 L to 10,000 mL, from 1,000 L to 10,000 L, from 2,000 L to 10,000 L, from 5,000 L to 10,000 L, from 7,500 L to 10,000 L, or greater than 10,000 L of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10. In some embodiments, systems and methods described herein including a chromatography column having beads of average diameter from 100 micrometers to 200 micrometers can process greater than 1 liter (L), greater than 100 L, greater than 200 L, greater than 500 L, greater than 1,000 L, greater than 2,000 L, greater than 5,000 L, greater than 7,500 L, or greater than 10,000 L of centrifuged, unfiltered fluid sample (e.g., a sample having a turbidity of from about 5.0 to about 500 Nephelometric Turbidity Units, NTUs), for example, wherein normalized chromatography column pressure (pressure normalized to column bed height (e.g., determined in megapascals of pressure per bed height in centimeters, MPa/BH(cm))) does not exceed 0.10.

In some embodiments, a system or method disclosed herein can achieve a percent recovery (e.g., yield recovery) of the substance of interest in the centrifuged sample after processing with the centrifugation unit, relative to the amount of substance of interest in the fluid sample when the fluid sample is introduced to the centrifugation unit, of from about 60% to about 70%, from about 70% to about 80%, from about 80% to about 90%, from about 90% to about 95%, from about 95% to about 99%, greater than 99%, up to about 60%, up to about 70%, up to about 80%, up to about 90%, up to about 95%, up to about 99%, at least about 75%, at least about 80%, at least about 85%, at least about 90%, or at least about 95%. In some embodiments, a system or method disclosed herein can achieve a percent recovery (e.g., yield recovery) of the substance of interest after processing with the chromatography unit, relative to the amount of substance of interest in the centrifuged sample when the centrifuged sample is introduced to the chromatography unit, of from about 60% to about 70%, from about 70% to about 80%, from about 80% to about 90%, from about 90% to about 95%, from about 95% to about 99%, greater than 99%, up to about 60%, up to about 70%, up to about 80%, up to about 90%, up to about 95%, up to about 99%, at least about 75%, at least about 80%, at least about 85%, at least about 90%, or at least about 95%. In some embodiments, a system or method disclosed herein can achieve a percent recovery (e.g., yield recovery) of the substance of interest after processing with the chromatography unit, relative to the amount of substance of interest in the fluid sample when the fluid sample is introduced to the centrifugation unit, of from about 60% to about 70%, from about 70% to about 80%, from about 80% to about 90%, from about 90% to about 95%, from about 95% to about 99%, greater than 99%, up to about 60%, up to about 70%, up to about 80%, up to about 90%, up to about 95%, up to about 99%, at least about 60%, at least about 70%, at least about 80%, at least about 90%, or at least about 95%.

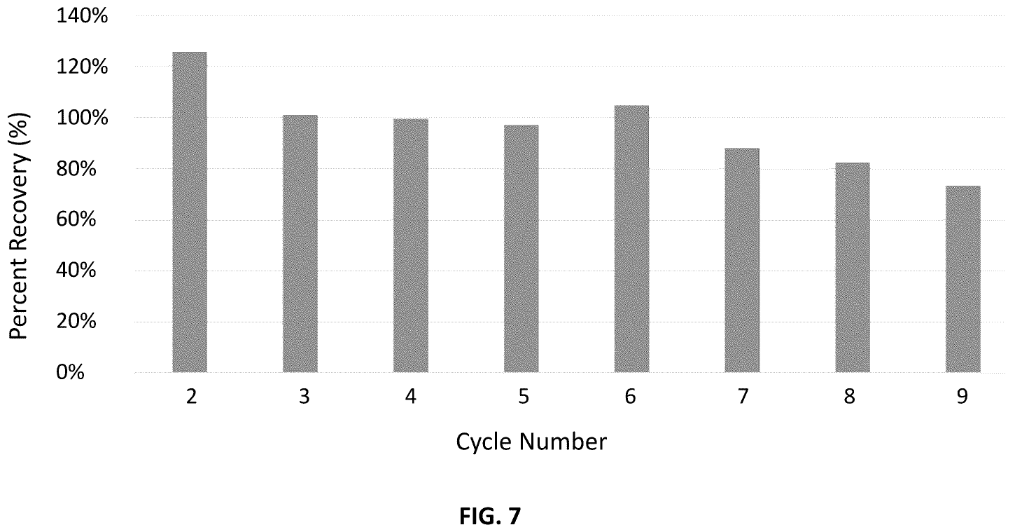

In some embodiments, systems and methods described herein can extend the useful life of a chromatography column. For example, systems and methods described herein can allow for a single chromatography column to be used for 2 or more cycles, 3 or more cycles, 4 or more cycles, 5 or more cycles, 6 or more cycles, 7 or more cycles, 8 or more cycles, 9 or more cycles, or 10 or more cycles (e.g., wherein each cycle includes processing at least about 20 mL of centrifuged, unfiltered fluid sample through the chromatography column). In some cases, a useful life of a chromatography column can be a period of usage during which the percent recovery of a substance of interest (e.g., after elution from the chromatography column) is at least 70%, at least 75%, at least 80%, at least 85%, at least 90%, at least 95%, at least 97%, or at least 99%. For example, systems and methods disclosed herein can maintain a percent recovery of a substance of interest from a single chromatography column of at least 70%, at least 75%, at least 80%, at least 85%, at least 90%, at least 95%, at least 97%, at least 99% for 2 or more cycles (e.g., wherein each cycle includes processing at least about 20 mL of centrifuged, unfiltered fluid sample through the chromatography column), in some embodiments. In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 3 or more cycles (e.g., wherein each cycle includes processing at least about 20 mL of centrifuged, unfiltered fluid sample through the chromatography column). In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 4 or more cycles (e.g., wherein each cycle includes processing a load of at least about 27 milligrams of the molecule of interest, as measured following centrifugation and prior to introduction into the chromatography unit, in centrifuged, unfiltered fluid sample through the chromatography column). In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 5 or more cycles (e.g., wherein each cycle includes processing at least about 20 mL of centrifuged, unfiltered fluid sample through the chromatography column). In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 6 or more cycles (e.g., wherein each cycle includes processing a load of at least about 27 milligrams of the molecule of interest, as measured following centrifugation and prior to introduction into the chromatography unit, in centrifuged, unfiltered fluid sample through the chromatography column). In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 7 or more cycles (e.g., wherein each cycle includes processing at least about 20 mL of centrifuged, unfiltered fluid sample through the chromatography column). In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 8 or more cycles (e.g., wherein each cycle includes processing a load of at least about 27 milligrams of the molecule of interest, as measured following centrifugation and prior to introduction into the chromatography unit, in centrifuged, unfiltered fluid sample through the chromatography column). In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 9 or more cycles (e.g., wherein each cycle includes processing a load of at least about 27 milligrams of the molecule of interest, as measured following centrifugation and prior to introduction into the chromatography unit, in centrifuged, unfiltered fluid sample through the chromatography column). In some embodiments, systems and methods disclosed herein can maintain a percent recovery of a substance of interest from a single chromatography column of at least 70%, at least 75%, at least 80%, at least 85%, at least 90%, at least 95%, at least 97%, at least 99% for 10 or more cycles (e.g., wherein each cycle includes processing at least about 20 mL of centrifuged, unfiltered fluid sample through the chromatography column). FIG. 7 shows percent recovery data for cycling experiments performed using the conditions indicated in Table 1 below, which shows data obtained during cycling tests used to determine a baseline column performance under typical use conditions. Cycle 0 in Table 1 shows an initial cycle performed to determine dynamic binding capacity of the column and to determine appropriate load amount for application to the column.

TABLE 1

| Cycle | | Load Applied |
|---|---|---|
| Number | Material | to Column |
| 0 | Depth Filtered Lysate | 45 mg |
| 1 | Centrifuged Lysate | 45 mg |
| 2 | Centrifuged Lysate | 27 mg |
| 3 | Centrifuged Lysate | 27 mg |
| 4 | Centrifuged Lysate | 27 mg |
| 5 | Centrifuged Lysate | 27 mg |
| Blank | Buffer | 0 mg |
| 6 | Centrifuged Lysate | 27 mg |
| 7 | Centrifuged Lysate | 27 mg |
| 8 | Centrifuged Lysate | 27 mg |
| 9 | Centrifuged Lysate | 27 mg |

The data in FIG. 7 shows that percent recovery was greater than 95% for each of cycles 2, 3, 4, 5, and 6 using centrifugation followed by affinity chromatography, as described herein. Cycles 7 and 8 showed a percent recovery of greater than 80%, and cycle 9 showed a percent recovery of greater than 75%. From these data, it is clear that the present systems and methods allow for extended use of chromatography units.

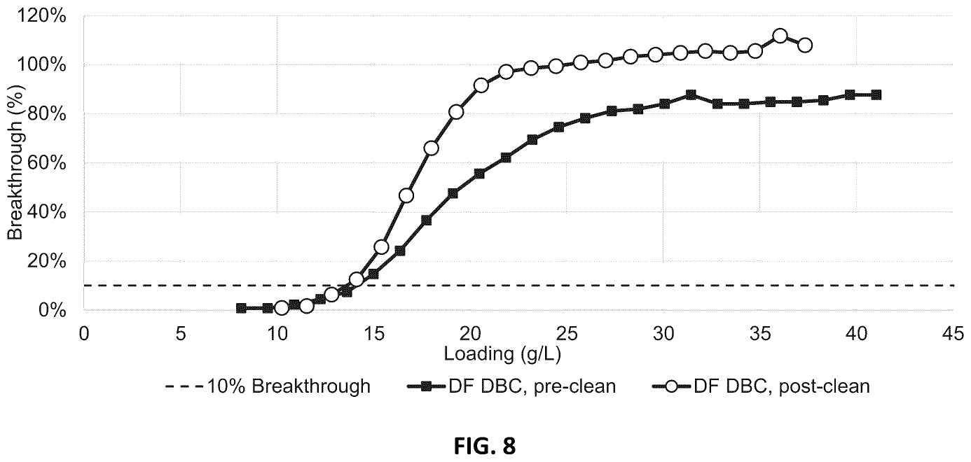

In some embodiments, washing the chromatography column (e.g., performing a wash step) between cycles can improve the performance of the system or method. A system or method disclosed herein can include one or more wash steps after a chromatography column cycle. A system or method disclosed herein can include one or more wash steps after a plurality of chromatography column cycles. A wash step can include flushing the chromatography column (and, optionally, one or more fluid pathways of the system) with one or more, two or more, three or more, four or more, five or more, six or more, seven or more, eight or more, nine or more, ten or more, fifteen or more, or twenty or more chromatography column volume's worth of wash fluid (e.g., chromatography buffer solution). In some embodiments, including one or more wash steps between cycles can remove contaminants from the column. Without being bound by theory, removal of contaminants from the chromatography column may reduce bioburden in the column and degradation of the affinity ligand(s) of the chromatography column beads (e.g., through protease activity). In some embodiments, including one or more wash steps between cycles of the chromatography column can improve load capacity of the chromatography column and/or improve the breakthrough kinetics of the chromatography column. FIG. 8 shows that the slope of the breakthrough curve for a chromatography column loaded with fluid sample subjected to depth filtration increases after washing, when the column has been used for 10 cycles. In some embodiments a wash solution can include one or more components selected from: Tris solution (e.g., 50 mM to 500 mM Tris solution, optionally pH-ed to 7.5, for example, 100 mM Tris solution, pH 7.5), urea (e.g., 1 molar (M) to 3 M urea, for example, 3 M urea), Polysorbate 20 (Tween 20) (e.g., 0.5% to 2% polysorbate, for example, 1% Polysorbate 20), arginine (e.g., 0.2 M to 1 M arginine, for example, 0.5 M arginine), isopropyl alcohol (IPA) (e.g., 5% to 20% isopropyl alcohol, for instance 10% IPA with 0.5M arginine, or 10% IPA with 3M urea and 1% Polysorbate 20), and/or sodium chloride (e.g., 0.1 M to 1 M sodium chloride, optionally pH'ed to 7.5, for example, 1 M sodium chloride, pH 7.5). In some cases, a method or system disclosed herein can include a strip wash step, wherein the chromatography column is flushed with a stripping solution (e.g., 0.1 normal (N) sodium hydroxide (NaOH)).

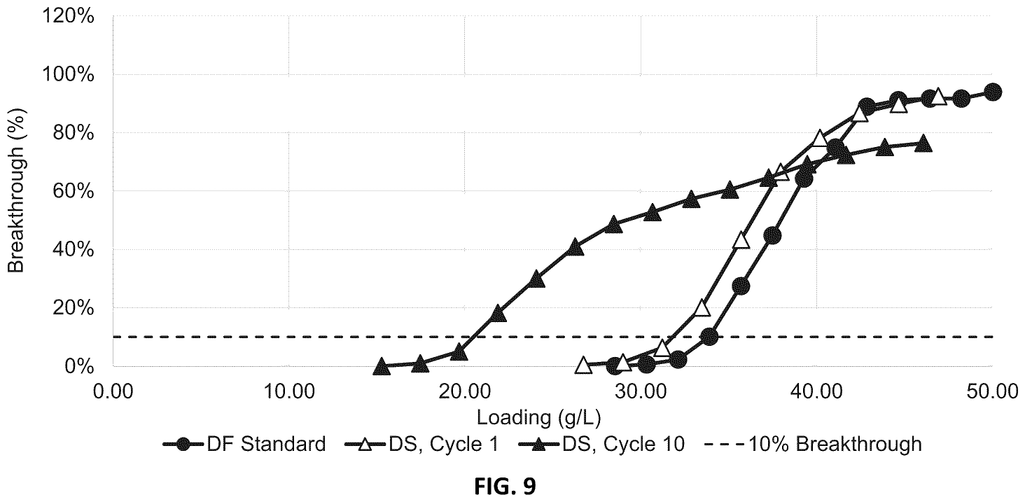

In some embodiments, systems and methods disclosed herein can improve breakthrough of substances of interest (e.g., molecules of interest), even at high fluid sample load concentrations and/or when columns are used repeatedly, indicating that systems and methods disclosed herein can exhibit increased load capacity compared to other technologies. FIG. 9 shows that systems and methods disclosed herein, where sample fluid is centrifuged and then loaded onto the chromatography column without filtering (black line with white triangular data points (“DS, cycle 1”)) reaches the 10% breakthrough threshold at approximately the same loading level (e.g., within a range of 31 g/L to 35 g/L) as existing protocols including depth filtration of fluid samples followed by the use of standard chromatography columns. As described above, the systems and methods disclosed herein can drastically decrease cost of system components and time to complete methods compared to those same existing protocols (black line with circular data points, (“DF Standard”)). Furthermore, FIG. 9 shows that the systems and methods described herein maintain a high load capacity, even when the same column is used 10 times without replacement or washing (black line with black triangular data points, (“DS, Cycle 10”)), wherein the system crosses the 10% breakthrough threshold at approximately 21 g/L. In some embodiments, systems and methods described herein including a chromatography column having beads of average diameter from 25 micrometers to 1,000 micrometers can process at least about 20 mL of unfiltered fluid sample including a load of from about 1.0 gram/liter (g/L) to about 40 grams/liter (g/L), from about 5.0 g/L to about 40 g/L, from about 10 g/L to about 40 g/L, from about 15 g/L to about 40 g/L, from about 20 g/L to about 40 g/L, from about 25 g/L to about 40 g/L, from about 30 g/L to about 40 g/L, from about 35 g/L to about 40 g/L, from about 10 g/L to about 35 g/L, from about 15 g/L to about 35 g/L, from about 20 g/L to about 35 g/L, from about 25 g/L to about 35 g/L, from about 30 g/L to about 35 g/L (e.g., a centrifuged, unfiltered sample having a turbidity of from about 5.0 to about 500 Nephelometric Turbidity Units, NTUs), for example, as evaluated by maintaining a breakthrough level of 10% or less (e.g., wherein breakthrough level reflects the percentage of the substance (e.g., molecule) of interest in the fluid that passes through the chromatography unit without being retained by the chromatography unit, as measured during a chromatography unit process cycle). In some embodiments, systems and methods described herein including a chromatography column having beads of average diameter from 50 micrometers to 100 micrometers can process at least about 20 mL of unfiltered fluid sample including a load of from about 1.0 gram/liter (g/L) to about 40 grams/liter (g/L), from about 5.0 g/L to about 40 g/L, from about 10 g/L to about 40 g/L, from about 15 g/L to about 40 g/L, from about 20 g/L to about 40 g/L, from about 25 g/L to about 40 g/L, from about 30 g/L to about 40 g/L, from about 35 g/L to about 40 g/L, from about 10 g/L to about 35 g/L, from about 15 g/L to about 35 g/L, from about 20 g/L to about 35 g/L, from about 25 g/L to about 35 g/L, from about 30 g/L to about 35 g/L (e.g., a centrifuged, unfiltered sample having a turbidity of from about 5.0 to about 500 Nephelometric Turbidity Units, NTUs), for example, as evaluated by maintaining a breakthrough level of 10% or less. In some embodiments, systems and methods described herein including a chromatography column having beads of average diameter from 100 micrometers to 200 micrometers can process at least about 20 mL of unfiltered fluid sample including a load of from about 1.0 gram/liter (g/L) to about 40 grams/liter (g/L), from about 5.0 g/L to about 40 g/L, from about 10 g/L to about 40 g/L, from about 15 g/L to about 40 g/L, from about 20 g/L to about 40 g/L, from about 25 g/L to about 40 g/L, from about 30 g/L to about 40 g/L, from about 35 g/L to about 40 g/L, from about 10 g/L to about 35 g/L, from about 15 g/L to about 35 g/L, from about 20 g/L to about 35 g/L, from about 25 g/L to about 35 g/L, from about 30 g/L to about 35 g/L (e.g., a centrifuged, unfiltered sample having a turbidity of from about 5.0 to about 500 Nephelometric Turbidity Units, NTUs), for example, as evaluated by maintaining a breakthrough level of 10% or less.

In some embodiments, a loading capacity of a centrifuged sample equal to or greater than the loading capacity of a depth filtered fluid sample. For example, a chromatographic unit of a system or method disclosed herein can have a loading capacity of at least 60%, at least 75%, at least 80%, at least 85%, at least 90%, at least 95%, or at least 100% (e.g., using a centrifuged sample, for instance, as described herein) as compared to a loading capacity of the chromatographic unit when loaded with a depth filtered fluid sample. In some embodiments, a chromatographic unit of a system or method disclosed herein can have a loading capacity of from 75% to 80%, from 80% to 85%, from 85% to 90%, from 90% to 95%, or from 95% to 100% (e.g., using a centrifuged sample, for instance, as described herein) as compared to a loading capacity of the chromatographic unit when loaded with a depth filtered fluid sample.

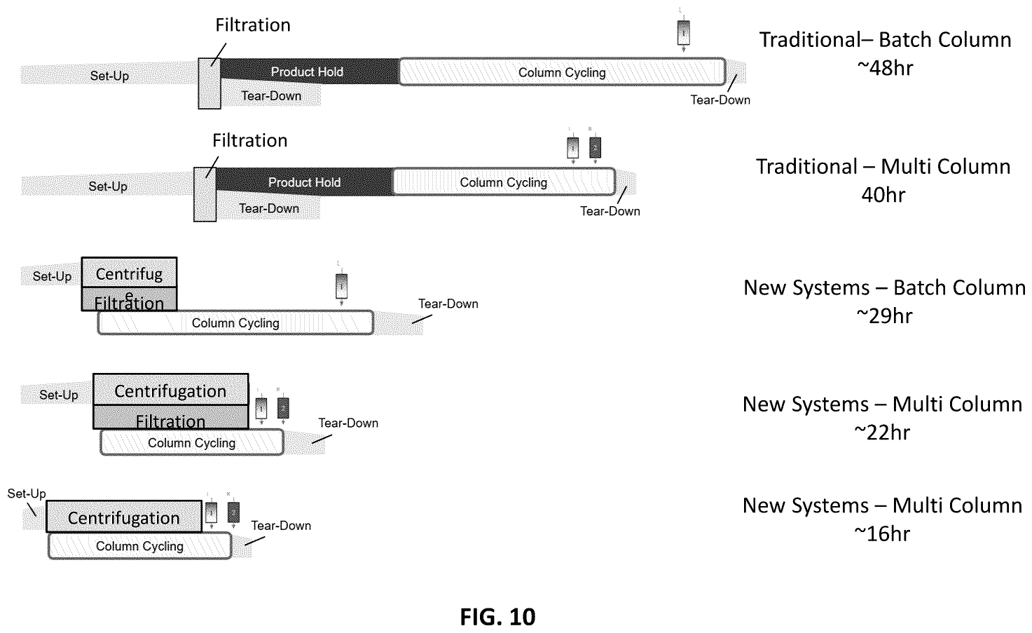

FIG. 10 shows a comparison of approximate time required to perform enrichment, purification, and/or isolation of substances of interest using traditional (e.g., existing) technology including depth filtration and affinity chromatography and using systems and methods described herein (e.g., “New systems”) for large scale processing applications (e.g., at least 50 L of fluid sample, at least 100 L of fluid sample, at least 200 L of fluid sample, at least 500 L of fluid sample, at least 750 L, at least 1,000 L of fluid sample, at least 2,000 L of fluid sample, at least 5,000 L of fluid sample, or at least 10,000 L of fluid sample). Use of batch (e.g., single column) processing in conjunction with depth filtration and affinity chromatography (“Traditional-Batch Column”) can require approximately 48 hours to complete, including set up, tear down, and logistical product hold times. Use of multi-column processing in conjunction with depth filtration and affinity chromatography (“Traditional-Multi-Column”) can require approximately 40 hours to complete, including set up, tear down, and logistical product hold times. Batch processing utilizing centrifugation (and simultaneous filtration) and affinity chromatography (“New Systems-Batch Column”) can require far less time (approximately 29 hours), including set up and tear down. Multi-column processing utilizing centrifugation (and simultaneous filtration) and affinity chromatography (“New Systems-Multi Column”) can reduce time required even further (to about 22 hours), including set up and tear down. Multi-column processing utilizing centrifugation and affinity chromatography can reduce time required even further (to about 16 hours), including set up and tear down. In some embodiments, a method or system disclosed herein can be configured to process at least 50 L, at least 200 L, at least 300 L, at least 400 L, at least 500 L, at least 600 L, at least 700 L, at least 800 L, at least 900 L, at least 1,000 L, at least 2,000 L, at least 5,000 L, or at least 10,000 L of fluid sample including a substance of interest with the centrifuge unit and the chromatography column in less than 40 hours, less than 35 hours, less than 30 hours, less than 25 hours, less than 24 hours, less than 20 hours, less than 18 hours, or less than 15 hours. For example, a system or method disclosed herein can be configured to process at least 2,000 L of the substance of interest with the centrifuge unit and the chromatography unit in less than 40 hours, less than 35 hours, less than 30 hours, less than 25 hours, less than 24 hours, less than 20 hours, less than 18 hours, or less than 15 hours. In some embodiments, a system or method disclosed herein can be configured to process at least 1,000 L of the substance of interest with the centrifuge unit and the chromatography unit in less than 40 hours, less than 35 hours, less than 30 hours, less than 25 hours, less than 24 hours, less than 20 hours, less than 18 hours, less than 15 hours, less than 12 hours, or less than 10 hours.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents can be resorted to, falling within the scope of the disclosure.

1. A system for separating a substance of interest of an unfiltered fluid sample comprising:
a centrifuge unit; and
a chromatography unit comprising a plurality of beads, each of the plurality of beads comprising a capture ligand; and
a fluidic apparatus configured to transfer the fluid sample after centrifugation from the centrifuge unit to the chromatography unit.

2. The system of claim 1, wherein each of the plurality of beads has a diameter of less than 200 micrometers.

3. A system for separating a substance of interest of an unfiltered fluid sample comprising:
a centrifuge unit; and
a chromatography unit in fluid communication with the centrifuge,
wherein the chromatography unit is configured to receive an unfiltered supernatant comprising the substance of interest from the centrifuge unit without the supernatant passing through a filter.

4. The system of claim 3, wherein the chromatography unit is an affinity chromatography column.

5. The system of claim 4, wherein the affinity chromatography column comprises a plurality of beads, each of the plurality of beads comprising a capture ligand.

6. The system of claim 5, wherein the capture ligand is protein A.

7. The system of claim 5, wherein the capture ligand is an adeno-associated virus ligand.

8. The system of claim 4, wherein the affinity chromatography column is configured to capture a molecule of interest selected from a protein, a carbohydrate, a lipid, or a nucleic acid.

9. The system of claim 1, wherein the system does not comprise a filter between the centrifuge unit and the chromatography unit.

10. The system of claim 5, wherein each of the plurality of beads has a diameter of from 50 micrometers to 100 micrometers or from 100 micrometers to 190 micrometers.

11. The system of claim 1, wherein the chromatography unit is configured as an axial flow column (AFC) or a radial flow column (RFC).

12. The system of claim 1, wherein the chromatography unit has a void volume fraction of 10% to 20%, 20% to 30%, 30% to 40%, or up to 50%.

13. The system of claim 1, wherein the system is a closed system.

14. The system of claim 3, wherein the system is configured to maintain sterility of the substance of interest by avoiding external contaminants.

15. The system of claim 1, wherein the centrifuge unit comprises an inlet tube configured to receive the fluid sample.

16. The system of claim 1, further comprising a pump configured to transfer fluid from the centrifuge unit to the chromatography unit.

17.-36. (canceled)

37. A method for collecting a substance of interest comprising:
centrifuging a fluid sample comprising the substance and a cellular component, and collecting a centrifuged fluid sample;
transferring the centrifuged fluid sample to a chromatography unit without filtering the centrifuged fluid sample; and
separating the substance of interest from the fluid sample using the chromatography unit.

38. The method of claim 37, further comprising washing the fluid sample after transferring the fluid sample to the chromatography unit.

39. The method of claim 37, wherein the fluid sample is washed two or more times after transferring the fluid sample to the chromatography unit.

40. The method of claim 37, wherein the fluid sample is washed with a solution comprising one or more mixture selected from:
5% to 20% isopropyl alcohol and 0.2 molar (M) to 1 M arginine;
5% to 20% isopropyl alcohol, 1 M to 3 M urea, and 0.5% to 2% polysorbate 20;
50 mM to 500 mM Tris, 0.1 M to 1 M sodium chloride; or
50 M to 500 mM Tris.

41.-119. (canceled)
